(19) United States
(12) Patent Application Publication
(10) Pub. No.: US 2023/0014358 A1
(43) Pub. Date: Jan. 19, 2023

(54) COMPOSITION FOR ANTICANCER TREATMENT, COMPRISING NK CELLS AND FUSION PROTEIN WHICH COMPRISES IL-2 PROTEIN AND CD80 PROTEIN

(71) Applicant: GI CELL, INC., Gyeonggi-do (KR)

(72) Inventors: Myoung Ho JANG, Seoul (KR); Chun-Pyo HONG, Gyeonggi-do (KR); Zung Yoon YANG, Incheon (KR); Young Jun KOH, Seoul (KR); June Sub LEE, Gyeonggi-do (KR); Young Joo CHOI, Gyeonggi-do (KR)

(21) Appl. No.: 17/778,168

(22) PCT Filed: Nov. 26, 2020
(86) PCT No.: PCT/KR2020/016949

§ 371 Date: May 19, 2022

(30) Foreign Application Priority Data
Nov. 27, 2019 (KR) 10-2019-0154631
Feb. 10, 2020 (KR) 10-2020-0015808

(51) Int. Cl. A61K 38/20; A61P 35/00; A61K 38/17; A61K 35/17

(52) U.S. Cl. CPC A61K 38/2013; A61P 35/00; A61K 38/1774; A61K 35/17

(57) ABSTRACT

Provided is an anticancer agent, comprising, as active ingredients, NK cells and a fusion protein which comprises an IL-2 protein and CD80 protein. In one specific embodiment, a fusion protein comprising a CD80 fragment, an immunoglobulin Fc and an IL-2 variant can activate immunocytes such as natural killer cells. In addition, since cancer can be effectively inhibited when co-administering with natural killer cells, the pharmaceutical composition increases the immune activity in the body so as to be effectively usable for cancer, there by having high industrial applicability.

### TECHNICAL FIELD

The present invention relates to a pharmaceutical composition for treating cancer including, as active ingredients, a fusion protein comprising a CD80 protein and an IL-2 variant, and a NK cell.

### BACKGROUND ART

IL-2, also called as T-cell growth factor (TCGF), is a globular glycoprotein that plays a central role in production, survival, and homeostasis of lymphocyte. IL-2 protein has a size of 15.5 kDa to 16 kDa and consists of 133 amino acids. IL-2 mediates various immune actions by binding to the IL-2 receptor which has three distinct subunits. In addition, IL-2 is synthesized mainly by activated T cells, in particular by CD4+ helper T cells. IL-2 stimulates proliferation and differentiation of T cells, and induces production of cytotoxic T lymphocytes (CTLs) and differentiation of peripheral blood lymphocytes into cytotoxic cells and lymphokine-activated killer cells (LAK cells).

Meanwhile, CD80, also known as B7-1, is a member of the B7 family of membrane-bound proteins that are involved in immune regulation by binding to its ligand by way of delivering costimulatory responses and coinhibitory responses.

CD80 is a transmembrane protein expressed on the surface of T cells, B cells, dendritic cells, and monocytes. CD80 is known to bind CD28, CTLA4 (CD152), and PD-L1. CD80, CD86, CTLA4, and CD28 are involved in a costimulatory-coinhibitory system. For example, they regulate activity of T cells and are involved in proliferation, differentiation, and survival thereof.

In addition, natural killer cells (hereinafter, NK cells) are known to exhibit anticancer activity by removing cancer cells (Loris Zamai et.al., J. Immunol., 178:4011-4016, 2007). The activity of NK cells is regulated by a balance of various activating and inhibitory receptor signaling. It is known that the anticancer activity of NK cells is also achieved by discriminating cancer cells through various immune receptors present on the surface. Due to major histocompatibility complex (MHC) class I present in normal cells, the normal cells are recognized by the inhibitory receptors of NK cells and not attacked thereby, but cancer cells or some infected cells are eliminated by NK cells due to reduced MHC Class I or ligands for activating receptors of NK cells. NK cells can eliminate cancer stem cells in addition to cancer cells, and thus are in the spotlight as a source for therapeutics that can not only inhibit the development, proliferation, and metastasis of cancer, but also reduce recurrence of cancer after complete recovery.

### DETAILED DESCRIPTION OF THE INVENTION

### Technical Problem

Accordingly, as a result of studying to develop a safe and effective IL-2, the present inventors found out that co-administration of a novel fusion protein dimer comprising an IL-2 protein and a CD80 protein in one molecule in combination with natural killer cells exhibits an excellent anticancer effect, and have completed the present invention.

### Solution to Problem

To achieve the above purpose, in accordance with one aspect of the present invention, there is provided an anticancer agent including, as active ingredients, a fusion protein dimer comprising an IL-2 protein and a CD80 protein, and a natural killer cell.

### Effects of the Invention

It was confirmed that a fusion protein dimer comprising an IL-2 protein and a CD80 protein may not only activate immune cells, but also exhibit synergistic effects when administered in combination with natural killer cells. Therefore, such combination therapy can be usefully applied to the treatment of cancer.

### BRIEF DESCRIPTION OF DRAWINGS

FIG. 1 is a schematic diagram of an embodiment of a fusion protein dimer used in the present invention;

FIG. 2 shows an image of SDS-PAGE confirming the obtained fusion protein dimer (GI-101);

FIG. 3 shows size exclusion chromatography (SEC) analysis of the obtained fusion protein dimer (GI-101);

FIG. 4 shows an image of SDS-PAGE confirming the obtained Fc-IL2v2 fusion protein;

FIG. 5 shows size exclusion chromatography (SEC) analysis of the obtained Fc-IL2v2 fusion protein;

FIG. 6 shows an image of SDS-PAGE confirming the obtained hCD80-Fc fusion protein;

FIG. 7 shows size exclusion chromatography (SEC) analysis of the obtained hCD80-Fc fusion protein;

FIG. 8 shows the results of the cancer cell viability according to treatment with GI-101 or CD80-Fc+Fc-IL2v2 when culturing a K562 cell line alone without natural killer cells (E/T ratio=0/1). In this case, E indicates an NK cell as an effector cell, and T indicates a K562 cancer cell line as a target cell.

FIG. 9 shows the results of the cancer cell viability according to treatment with GI-101 or CD80-Fc+Fc-IL2v2 when culturing an MDA-MB-231 cell line alone without natural killer cells (E/T ratio=0/1). In this case, E indicates an NK cell as an effector cell, and T indicates an MDA-MB-231 cancer cell line as a target cell.

FIG. 10 shows the results of the cancer cell viability according to treatment with GI-101 or CD80-Fc+Fc-IL2v2 when culturing an HCT-116 cell line alone without natural killer cells (E/T ratio=0/1). In this case, E indicates an NK cell as an effector cell, and T indicates an HCT-116 cancer cell line as a target cell.

FIG. 11 shows the results of the cancer cell viability according to treatment with GI-101 or CD80-Fc+Fc-IL2v2 when culturing a A549 cell line alone without natural killer cells (E/T ratio=0/1). In this case, E indicates an NK cell as an effector cell, and T indicates a A549 cancer cell line as a target cell.

FIG. 12 shows the results of the cancer cell viability according to treatment with a combination material GI-101 or CD80-Fc+Fc-IL2v2 when co-culturing a K562 cell line (target cell) with natural killer cells (effector cell) in E/T ratio=1/3.

Here, Y-axis=0 indicates that the range of the viability signal (red signal) of cancer cells at the time of initial seeding is set to as 0. Y-axis>0 is the case where the range of viability signal of cancer cells found after the time of seeding increases, indicating that cell proliferation rate is faster than the cell death rate (cell proliferation rate>cell death rate). That is, it can be seen as increase in the number of cancer cells. However, it is thought that the lower the value of increase amount, the more inhibited the proliferation of cancer cells. Meanwhile, if the area of the viable cancer cells measured is smaller than that of the viable cancer cells determined at the time of initial seeding, it is expressed as a negative value, which indicates that the cell proliferation rate is slower than the cell death rate (cell proliferation rate<cell death rate). That is, decrease in the area compared to that measured at the time of initial seeding is expressed as a negative value. Therefore, it is suggested that a larger negative value may be related to not only inhibition of proliferation of cancer cells but also death of cancer cells thereof.

FIG. 13 shows the results of the cancer cell viability according to treatment with a combination material GI-101 or CD80-Fc+Fc-IL2v2 when co-culturing a K562 cell line with natural killer cells in E/T ratio=1/1.

FIG. 14 shows the results of the cancer cell viability according to treatment with a combination material GI-101 or CD80-Fc+Fc-IL2v2 when co-culturing a K562 cell line with natural killer cells in E/T ratio=3/1.

FIG. 15 shows the results of the cancer cell viability according to treatment with a combination material GI-101 or CD80-Fc+Fc-IL2v2 when co-culturing a K562 cell line with natural killer cells in E/T ratio=10/1.

FIG. 16 shows the results of the cancer cell viability according to treatment with a combination material GI-101 or CD80-Fc+Fc-IL2v2 when co-culturing a MDA-MB231 cell line (target cell) with natural killer cells (effector cell) in E/T ratio=1/3.

FIG. 17 shows the results of the cancer cell viability according to treatment with a combination material GI-101 or CD80-Fc+Fc-IL2v2 when co-culturing a MDA-MB231 cell line with natural killer cells in E/T ratio=1/1.

FIG. 18 shows the results of the cancer cell viability according to treatment with a combination material GI-101 or CD80-Fc+Fc-IL2v2 when co-culturing a MDA-MB231 cell line with natural killer cells in E/T ratio=3/1.

FIG. 19 shows the results of the cancer cell viability according to treatment with a combination material GI-101 or CD80-Fc+Fc-IL2v2 when co-culturing a MDA-MB231 cell line with natural killer cells in E/T ratio=10/1.

FIG. 20 shows the results of the cancer cell viability according to treatment with a combination material GI-101 or CD80-Fc+Fc-IL2v2 when co-culturing a HCT-116 cell line (target cell) with natural killer cells (effector cell) in E/T ratio=1/3.

FIG. 21 shows the results of the cancer cell viability according to treatment with a combination material GI-101 or CD80-Fc+Fc-IL2v2 when co-culturing a HCT-116 cell line with natural killer cells in E/T ratio=1/1.

FIG. 22 shows the results of the cancer cell viability according to treatment with a combination material GI-101 or CD80-Fc+Fc-IL2v2 when co-culturing a HCT-116 cell line with natural killer cells in E/T ratio=3/1.

FIG. 23 shows the results of the cancer cell viability according to treatment with a combination material GI-101 or CD80-Fc+Fc-IL2v2 when co-culturing a HCT-116 cell line with natural killer cells in E/T ratio=10/1.

FIG. 24 shows the results of the cancer cell viability according to treatment with a combination material GI-101 or CD80-Fc+Fc-IL2v2 when co-culturing a A549 cell line (target cell) with natural killer cells (effector cell) in E/T ratio=1/3.

FIG. 25 shows the results of the cancer cell viability according to treatment with a combination material GI-101 or CD80-Fc+Fc-IL2v2 when co-culturing a A549 cell line with natural killer cells in E/T ratio=1/1.

FIG. 26 shows the results of the cancer cell viability according to treatment with a combination material GI-101 or CD80-Fc+Fc-IL2v2 when co-culturing a A549 cell line with natural killer cells in E/T ratio=3/1.

FIG. 27 shows the results of the cancer cell viability according to treatment with a combination material GI-101 or CD80-Fc+Fc-IL2v2 when co-culturing a A549 cell line with natural killer cells in E/T ratio=10/1.

FIG. 28 is a schematic diagram showing an administration schedule of mGI-101 and/or NK cells combination therapies to a CT26 transplanted carcinoma mouse model.

FIG. 29 shows the results of the tumor growth inhibitory effect according to co-administration of natural killer cells and mGI-101 to a CT26 transplanted carcinoma mouse model.

FIG. 30 shows the percentage of the tumor growth inhibition according to co-administration of natural killer cells and mGI-101 to a CT26 transplanted carcinoma mouse model.

FIG. 31 shows the tumor growth measurements of individual laboratory animals in each treatment group (vehicle, NK cell, mGI-101, NK cell+mGI-101) in a CT26 transplanted carcinoma mouse model.

FIG. 32 shows the tumor growth measurements of individual laboratory animals in the vehicle group in a CT26 transplanted carcinoma mouse model.

FIG. 33 shows the tumor growth measurements of individual laboratory animals in the NK cell treatment group in a CT26 transplanted carcinoma mouse model.

FIG. 34 shows the tumor growth measurements of individual laboratory animals in the mGI-101 treatment group in a CT26 transplanted carcinoma mouse model.

FIG. 35 shows the tumor growth measurements of individual laboratory animals in the co-administration group of natural killer cells and mGI-101 in a CT26 transplanted carcinoma mouse model.

### BEST MODE FOR CARRYING OUT THE INVENTION

An aspect of the present invention provides a pharmaceutical composition including, as active ingredients, a fusion protein comprising a CD80 protein and an IL-2 protein, and NK cells.

Another aspect of the present invention provides a pharmaceutical composition for treating cancer including, as active ingredients, a fusion protein comprising a CD80 protein and an IL-2 protein, and NK cells.

### Natural Killer Cell

As used herein, the term “NK cell” refers to a natural killer cell (hereinafter NK cell), and is one of innate immune cells which directly interacts with various macrophages and T cells or generates cytokines to regulate immune responses, and thereby playing an important role in autoimmune diseases. In the present invention, NK cells may be isolated from the spleen or bone marrow, but are not limited thereto. Specifically, the NK cells may be obtained from autologous or heterologous cells.

In addition, the NK cells may be derived from mammals or humans. Preferably, it may be obtained from an individual who intends to receive NK cell treatment. In this case, the NK cells may be directly isolated from blood of an individual and used, or immature NK cells or stem cells obtained from the individual may be differentiated and used.

In addition, the natural killer cells may be obtained by the following steps including: i) isolating cells that do not express CD3 from peripheral blood mononuclear cells (PBMC); ii) isolating cells that express CD56 from cells that do not express CD3 isolated in the above step; and iii) culturing an isolated cell in the presence of a fusion protein dimer comprising IL-2 or a variant thereof and CD80 or a fragment thereof.

Further, the natural killer cells may be obtained by the following steps including: i) isolating cells that do not express CD3 from PBMCs; and ii) culturing the isolated cells in the presence of a fusion protein dimer comprising IL-2 or a variant thereof and CD80 or a fragment thereof.

Furthermore, the natural killer cells may be obtained by the following steps including: i) isolating cells that do express CD56 from PBMCs; and ii) culturing the isolated cells in the presence of a fusion protein dimer comprising IL-2 or a variant thereof and CD80 or a fragment thereof.

A Fusion Protein Dimer Comprising an IL-2 Protein and a CD80 Protein

As used herein, the term “IL-2” or “interleukin-2”, unless otherwise stated, refers to any wild-type IL-2 obtained from any vertebrate source, including mammals, for example, primates (such as humans) and rodents (such as mice and rats). IL-2 may be obtained from animal cells, and also includes one obtained from recombinant cells capable of producing IL-2. In addition, IL-2 may be wild-type IL-2 or a variant thereof.

In the present specification, IL-2 or a variant thereof may be collectively expressed by the term “IL-2 protein” or “IL-2 polypeptide.” IL-2, an IL-2 protein, an IL-2 polypeptide, and an IL-2 variant specifically bind to, for example, an IL-2 receptor. This specific binding may be identified by methods known to those skilled in the art.

An embodiment of IL-2 may have the amino acid sequence of SEQ ID NO: 35 or SEQ ID NO: 36. Here, IL-2 may also be in a mature form. Specifically, the mature IL-2 may not comprise a signal sequence, and may have the amino acid sequence of SEQ ID NO: 10. Here, IL-2 may be used under a concept encompassing a fragment of wild-type IL-2 in which a portion of N-terminus or C-terminus of the wild-type IL-2 is truncated.

In addition, the fragment of IL-2 may be in a form in which 1, 2, 3, 4, 5, 6, 7, 8, 9, 10, 11, 12, 13, 14, 15, 16, 17, 18, 19, 20, 21, 22, 23, 24, or 25 continuous amino acids are truncated from N-terminus of a protein having the amino acid sequence of SEQ ID NO: 35 or SEQ ID NO: 36. In addition, the fragment of IL-2 may be in a form in which 1, 2, 3, 4, 5, 6, 7, 8, 9, 10, 11, 12, 13, 14, 15, 16, 17, 18, 19, 20, 21, 22, 23, 24, or 25 continuous amino acids are truncated from C-terminus of a protein having the amino acid sequence of SEQ ID NO: 35 or SEQ ID NO: 36.

As used herein, the term “IL-2 variant” refers to a form in which a portion of amino acids in the full-length IL-2 or the above-described fragment of IL-2 is substituted. That is, an IL-2 variant may have an amino acid sequence different from wild-type IL-2 or a fragment thereof. However, an IL-2 variant may have activity equivalent or similar to the wild-type IL-2. Here, “IL-2 activity” may, for example, refer to specific binding to an IL-2 receptor, which specific binding can be measured by methods known to those skilled in the art.

Specifically, an IL-2 variant may be obtained by substitution of a portion of amino acids in the wild-type IL-2. An embodiment of the IL-2 variant obtained by amino acid substitution may be obtained by substitution of at least one of the 38^{th}, 42^{nd}, 45^{th}, 61^{st}, and 72^{nd} amino acids in the amino acid sequence of SEQ ID NO: 10.

Specifically, the IL-2 variant may be obtained by substitution of at least one of the 38^{th}, 42^{nd}, 45^{th}, 61^{st}, or 72^{nd} amino acid in the amino acid sequence of SEQ ID NO: 10 with another amino acid. In addition, when IL-2 is in a form in which a portion of N-terminus in the amino acid sequence of SEQ ID NO: 35 is truncated, the amino acid at a position complementarily corresponding to that in the amino acid sequence of SEQ ID NO: 10 may be substituted with another amino acid. For example, when IL-2 has the amino acid sequence of SEQ ID NO: 35, its IL-2 variant may be obtained by substitution of at least one of 58^{th}, 62^{nd}, 65^{th}, 81^{st}, or 92^{nd} amino acid in the amino acid sequence of SEQ ID NO: 35 with another amino acid. These amino acid residues correspond to the 38^{th}, 42^{nd}, 45^{th}, 61^{st}, and 72^{nd} amino acid residues in the amino acid sequence of SEQ ID NO: 10, respectively. According to an embodiment, one, two, three, four, five, six, seven, eight, nine, or ten amino acids may be substituted as long as such IL-2 variant maintains IL-2 activity. According to another embodiment, one to five amino acids may be substituted.

In an embodiment, an IL-2 variant may be in a form in which two amino acids are substituted. Specifically, the IL-2 variant may be obtained by substitution of the 38^{th} and 42^{nd} amino acids in the amino acid sequence of SEQ ID NO: 10. In addition, in an embodiment, the IL-2 variant may be obtained by substitution of the 38^{th} and 45^{th} amino acids in the amino acid sequence of SEQ ID NO: 10. In addition, in an embodiment, the IL-2 variant may be obtained by substitution of the 38′ and 61^{st} amino acids in the amino acid sequence of SEQ ID NO: 10. In addition, in an embodiment, the IL-2 variant may be obtained by substitution of the 38^{th} and 72^{nd} amino acids in the amino acid sequence of SEQ ID NO: 10. In addition, in an embodiment, the IL-2 variant may be obtained by substitution of the 42^{nd} and 45^{th} amino acids in the amino acid sequence of SEQ ID NO: 10. In addition, in an embodiment, the IL-2 variant may be obtained by substitution of the 42^{nd} and 61^{st} amino acids in the amino acid sequence of SEQ ID NO: 10. In addition, in an embodiment, the IL-2 variant may be obtained by substitution of the 42^{nd} and 72^{nd} amino acids in the amino acid sequence of SEQ ID NO: 10. In addition, in an embodiment, the IL-2 variant may be obtained by substitution of the 45^{th} and 61^{st} amino acids in the amino acid sequence of SEQ ID NO: 10. In addition, in an embodiment, the IL-2 variant may be obtained by substitution of the 45^{th} and 72^{nd} amino acids in the amino acid sequence of SEQ ID NO: 10. In addition, in an embodiment, the IL-2 variant may be obtained by substitution of the 61^{st} and 72^{nd} amino acids in the amino acid sequence of SEQ ID NO: 10.

Furthermore, an IL-2 variant may be in a form in which three amino acids are substituted. Specifically, the IL-2 variant may be obtained by substitution of the 38^{th}, 42^{nd}, and 45^{th} amino acids in the amino acid sequence of SEQ ID NO: 10. In addition, in an embodiment, the IL-2 variant may be obtained by substitution of the 38^{th}, 42^{nd}, and 61^{st} amino acids in the amino acid sequence of SEQ ID NO: 10. In addition, in an embodiment, the IL-2 variant may be obtained by substitution of the 38^{th}, 42^{nd}, and 72^{nd} amino acids in the amino acid sequence of SEQ ID NO: 10. In addition, in an embodiment, the IL-2 variant may be obtained by substitution of the 38^{th}, 45th, and 61^{st} amino acids in the amino acid sequence of SEQ ID NO: 10. In addition, in an embodiment, the IL-2 variant may be obtained by substitution of the 38^{th}, 45th, and 72^{nd} amino acids in the amino acid sequence of SEQ ID NO: 10. In addition, in an embodiment, the IL-2 variant may be obtained by substitution of the 38^{th}, 61^{st}, and 72^{nd} amino acids in the amino acid sequence of SEQ ID NO: 10. In addition, in an embodiment, the IL-2 variant may be obtained by substitution of the 42^{nd}, 45^{th}, and 61^{st} amino acids in the amino acid sequence of SEQ ID NO: 10. In addition, in an embodiment, the IL-2 variant may be obtained by substitution of the 42^{nd}, 45th, and 72^{nd} amino acids in the amino acid sequence of SEQ ID NO: 10. In addition, in an embodiment, the IL-2 variant may be obtained by substitution of the 45^{th}, 61^{st}, and 72^{nd} amino acids in the amino acid sequence of SEQ ID NO: 10.

In addition, an IL-2 variant may be in a form in which four amino acids are substituted. Specifically, the IL-2 variant may be obtained by substitution of the 38^{th}, 42^{nd}, 45^{th}, and 61^{st} amino acids in the amino acid sequence of SEQ ID NO: 10. In addition, in an embodiment, the IL-2 variant may be obtained by substitution of the 38^{th}, 42^{nd}, 45^{th}, and 72^{nd} amino acids in the amino acid sequence of SEQ ID NO: 10. In addition, in an embodiment, the IL-2 variant may be obtained by substitution of the 38^{th}, 45^{th}, 61^{st}, and 72^{nd} amino acids in the amino acid sequence of SEQ ID NO: In addition, in an embodiment, the IL-2 variant may be obtained by substitution of the 38^{th}, 42^{nd} 61^{st} and 72^{nd} amino acids in the amino acid sequence of SEQ ID NO: 10. In addition, in an embodiment, the IL-2 variant may be obtained by substitution of 42^{nd}, 45^{th} 61^{st} and 72^{nd} amino acids in the amino acid sequence of SEQ ID NO: 10.

Furthermore, an IL-2 variant may be in a form in which five amino acids are substituted. Specifically, the IL-2 variant may be obtained by substitution of each of the 38^{th}, 42^{nd}, 45^{th}, 61^{st}, and 72^{nd} amino acids in the amino acid sequence of SEQ ID NO: 10 with another amino acid.

Here, the “another amino acid” introduced by the substitution may be any one selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. However, regarding amino acid substitution for the IL-2 variant, in the amino acid sequence of SEQ ID NO: 10, the 38^{th} amino acid cannot be substituted with arginine, the 42^{nd} amino acid cannot be substituted with phenylalanine, the 45^{th} amino acid cannot be substituted with tyrosine, the 61^{st} amino acid cannot be substituted with glutamic acid, and the 72^{nd} amino acid cannot be substituted with leucine.

Regarding amino acid substitution for an IL-2 variant, in the amino acid sequence of SEQ ID NO: 10, the 38^{th} amino acid, arginine, may be substituted with an amino acid other than arginine. Preferably, regarding amino acid substitution for an IL-2 variant, in the amino acid sequence of SEQ ID NO: 10, the 38^{th} amino acid, arginine, may be substituted with alanine (R38A).

Regarding amino acid substitution for an IL-2 variant, in the amino acid sequence of SEQ ID NO: 10, the 42^{nd} amino acid, phenylalanine, may be substituted with an amino acid other than phenylalanine. Preferably, regarding amino acid substitution for an IL-2 variant, in the amino acid sequence of SEQ ID NO: 10, the 42^{nd} amino acid, phenylalanine, may be substituted with alanine (F42A).

Regarding amino acid substitution for an IL-2 variant, in the amino acid sequence of SEQ ID NO: 10, the 45^{th} amino acid, tyrosine, may be substituted with an amino acid other than tyrosine. Preferably, regarding amino acid substitution for an IL-2 variant, in the amino acid sequence of SEQ ID NO: 10, the 45^{th} amino acid, tyrosine, may be substituted with alanine (Y45A).

Regarding amino acid substitution for an IL-2 variant, in the amino acid sequence of SEQ ID NO: 10, the 61^{st} amino acid, glutamic acid, may be substituted with an amino acid other than glutamic acid. Preferably, regarding amino acid substitution for an IL-2 variant, in the amino acid sequence of SEQ ID NO: 10, the 61^{st} amino acid, glutamic acid, may be substituted with arginine (E61R). Regarding amino acid substitution for an IL-2 variant, in the amino acid sequence of SEQ ID NO: 10, the 72^{nd} amino acid, leucine, may be substituted with an amino acid other than leucine. Preferably, regarding amino acid substitution for an IL-2 variant, in the amino acid sequence of SEQ ID NO: 10, the 72^{nd} amino acid, leucine, may be substituted with glycine (L72G).

Specifically, an IL-2 variant may be obtained by at least one substitution selected from the group consisting of R38A, F42A, Y45A, E61R, and L72G, in the amino acid sequence of SEQ ID NO: 10.

Specifically, an IL-2 variant may be obtained by amino acid substitutions at two, three, four, or five positions among the positions selected from the group consisting of R38A, F42A, Y45A, E61R, and L72G.

In addition, an IL-2 variant may be in a form in which two amino acids are substituted. Specifically, an IL-2 variant may be obtained by the substitutions, R38A and F42A. In addition, in an embodiment, an IL-2 variant may be obtained by the substitutions, R38A and Y45A. In addition, in an embodiment, an IL-2 variant may be obtained by the substitutions, R38A and E61R. In addition, in an embodiment, an IL-2 variant may be obtained by the substitutions, R38A and L72G. In addition, in an embodiment, an IL-2 variant may be obtained by the substitutions, F42A and Y45A. In addition, in an embodiment, an IL-2 variant may be obtained by the substitutions, F42A and E61R. In addition, in an embodiment, an IL-2 variant may be obtained by the substitutions, F42A and L72G. In addition, in an embodiment, an IL-2 variant may be obtained by the substitutions, E61R and L72G.

Furthermore, an IL-2 variant may be in a form in which three amino acids are substituted. Specifically, an IL-2 variant may be obtained by the substitutions, R38A, F42A, and Y45A. In addition, in an embodiment, an IL-2 variant may be obtained by the substitutions, R38A, F42A, and E61R. In addition, in an embodiment, an IL-2 variant may be obtained by the substitutions, R38A, F42A, and L72G. In addition, in an embodiment, an IL-2 variant may be obtained by the substitutions, R38A, Y45A, and E61R. In addition, in an embodiment, an IL-2 variant may be obtained by the substitutions, R38A, Y45A, and L72G. In addition, in an embodiment, an IL-2 variant may be obtained by the substitutions, F42A, Y45A, and E61R. In addition, in an embodiment, an IL-2 variant may be obtained by the substitutions, F42A, Y45A, and L72G. In addition, in an embodiment, an IL-2 variant may be obtained by the substitutions, F42A, E61R, and L72G. In addition, in an embodiment, an IL-2 variant may be obtained by the substitutions, Y45A, E61R, and L72G.

In addition, an IL-2 variant may be in a form in which four amino acids are substituted. Specifically, an IL-2 variant may be obtained by the substitutions, R38A, F42A, Y45A, and E61R. In addition, in an embodiment, an IL-2 variant may be obtained by the substitutions, R38A, F42A, Y45A, and L72G. In addition, in an embodiment, an IL-2 variant may be obtained by the substitutions, R38A, F42A, E61R, and L72G. In addition, in an embodiment, an IL-2 variant may be obtained by the substitutions, R38A, Y45A, E61R, and L72G. In addition, in an embodiment, an IL-2 variant may be obtained by the substitutions, F42A, Y45A, E61R, and L72G.

Furthermore, an IL-2 variant may be obtained by the substitutions, R38A, F42A, Y45A, E61R, and L72G.

Preferably, an embodiment of the IL-2 variant may comprise which are any one selected from the following substitution combinations (a) to (d) in the amino acid sequence of SEQ ID NO: 10:

(a) R38A/F42A;

(b) R38A/F42A/Y45A;

(c) R38A/F42A/E61R; or

(d) R38A/F42A/L72G.

Here, when IL-2 has the amino acid sequence of SEQ ID NO: 35, an amino acid substitution may be present at a position complementarily corresponding to that in the amino acid sequence of SEQ ID NO: 10. In addition, even when IL-2 is a fragment of the amino acid sequence of SEQ ID NO: 35, an amino acid substitution may be present at a position complementarily corresponding to that in the amino acid sequence of SEQ ID NO: 10.

Specifically, the IL-2 variant may have the amino acid sequence of SEQ ID NO: 6, 22, 23, or 24.

In addition, the IL-2 variant may be characterized by having low in vivo toxicity. Here, the low in vivo toxicity may be a side effect caused by binding of IL-2 to the IL-2 receptor alpha chain (IL-2Ru). Various IL-2 variants have been developed to ameliorate the side effect caused by binding of IL-2 to IL-2Ra, and such IL-2 variants may be those disclosed in U.S. Pat. No. 5,229,109 and Korean Patent No. 1667096. In particular, IL-2 variants described in the present application have low binding ability for the IL-2 receptor alpha chain (IL-2Ru) and thus have lower in vivo toxicity than the wild-type IL-2.

As used herein, the term “CD80”, also called “B7-1”, is a membrane protein present in dendritic cells, activated B cells, and monocytes. CD80 provides co-stimulatory signals essential for activation and survival of T cells. CD80 is known as a ligand for the two different proteins, CD28 and CTLA-4, present on the surface of T cells. CD80 consists of 288 amino acids, and may specifically have the amino acid sequence of SEQ ID NO: 11. In addition, as used herein, the term “CD80 protein” refers to the full-length CD80 or a CD80 fragment.

As used herein, the term “CD80 fragment” refers to a truncated form of CD80. In addition, the CD80 fragment may be an extracellular domain of CD80. An embodiment of the CD80 fragment may be obtained by deletion of the 1^{st} to 34^{th} amino acids from N-terminus which are a signal sequence of CD80. Specifically, an embodiment of the CD80 fragment may be a protein consisting of the 35^{th} to 288^{th} amino acids in SEQ ID NO: 11. In addition, an embodiment of the CD80 fragment may be a protein consisting of the 35^{th} to 242^{nd} amino acids in SEQ ID NO: 11. In addition, an embodiment of the CD80 fragment may be a protein consisting of the 35^{th} to 232^{nd} amino acids in SEQ ID NO: 11. In addition, an embodiment of the CD80 fragment may be a protein consisting of the 35^{th} to 139^{th} amino acids in SEQ ID NO: 11. In addition, an embodiment of the CD80 fragment may be a protein consisting of the 142^{nd} to 242^{nd} amino acids in SEQ ID NO: 11. In an embodiment, a CD80 fragment may have the amino acid sequence of SEQ ID NO: 2.

In addition, the IL-2 protein and the CD80 protein may be linked to each other via a linker or a carrier. Specifically, the IL-2 or a variant thereof and the CD80 (B7-1) or a fragment thereof may be linked to each other via a linker or a carrier. In the present description, the linker and the carrier may be used interchangeably.

The linker links two proteins. An embodiment of the linker may include 1 to 50 amino acids, albumin or a fragment thereof, an Fc domain of an immunoglobulin, or the like. Here, the Fc domain of immunoglobulin refers to a protein that comprises heavy chain constant region 2 (CH2) and heavy chain constant region 3 (CH3) of an immunoglobulin, and does not comprise heavy and light chain variable regions and light chain constant region 1 (CH1) of an immunoglobulin. The immunoglobulin may be IgG, IgA, IgE, IgD, o45r IgM, and may preferably be IgG4. Here, Fc domain of wild-type immunoglobulin G4 may have the amino acid sequence of SEQ ID NO: 4.

In addition, the Fc domain of an immunoglobulin may be an Fc domain variant as well as wild-type Fc domain. In addition, as used herein, the term “Fc domain variant” may refer to a form which is different from the wild-type Fc domain in terms of glycosylation pattern, has a high glycosylation as compared with the wild-type Fc domain, or has a low glycosylation as compared with the wild-type Fc domain, or a deglycosylated form. In addition, an aglycosylated Fc domain is included therein. The Fc domain or a variant thereof may be adapted to have an adjusted number of sialic acids, fucosylations, or glycosylations, through culture conditions or genetic engineering of a host.

In addition, glycosylation of the Fc domain of an immunoglobulin may be modified by conventional methods such as chemical methods, enzymatic methods, and genetic engineering methods using microorganisms. In addition, the Fc domain variant may be in a mixed form of respective Fc regions of immunoglobulins, IgG, IgA, IgE, IgD, and IgM. In addition, the Fc domain variant may be in a form in which some amino acids of the Fc domain are substituted with other amino acids. An embodiment of the Fc domain variant may have the amino acid sequence of SEQ ID NO: 12.

The fusion protein may have a structure in which, using an Fc domain as a linker (or carrier), a CD80 protein and an IL-2 protein, or an IL-2 protein and a CD80 protein are linked to N-terminus and C-terminus of the fusion protein (FIG. 1). Linkage between N-terminus or C-terminus of the Fc domain and CD-80 or IL-2 may optionally be achieved by a linker peptide.

Specifically, a fusion protein may consist of the following structural formula (I) or (II):

N′—X-[linker(1)]ₙ-Fc domain-[linker(2)]ₘ-Y—C′ (I)

N′—Y-[linker(1)]ₙ-Fc domain-[linker(2)]ₘ-X—C′ (II)

Here, in the structural formulas (I) and (II),

N′ is the N-terminus of the fusion protein,

C′ is the C-terminus of the fusion protein,

X is a CD80 protein,

Y is an IL-2 protein,

the linkers (1) and (2) are peptide linkers, and

n and m are each independently 0 or 1.

Preferably, the fusion protein may consist of the structural formula (I). The IL-2 protein is as described above. In addition, the CD80 protein is as described above. According to an embodiment, the IL-2 protein may be an IL-2 variant with one to five amino acid substitutions as compared with the wild-type IL-2. The CD80 protein may be a fragment obtained by truncation of up to about 34 continuous amino acid residues from the N-terminus or C-terminus of the wild-type CD80. Alternatively, the CD80 protein may be an extracellular immunoglobulin-like domain having the activity of binding to the T cell surface receptors CTLA-4 and CD28.

Specifically, the fusion protein may have the amino acid sequence of SEQ ID NO: 9, 26, 28, or 30. According to another embodiment, the fusion protein includes a polypeptide having a sequence identity of 85%, 86%, 87%, 88%, 89%, 90%, 91%, 92%, 93%, 94%, 95%, 96%, 97%, 98%, 99%, or 100% to the amino acid sequence of SEQ ID NO: 9, 26, 28, or 30. Here, the identity is, for example, percent homology, and may be determined through homology comparison software such as BlastN software of the National Center of Biotechnology Information (NCBI).

The peptide linker (1) may be included between the CD80 protein and the Fc domain. The peptide linker (1) may consist of 5 to 80 continuous amino acids, 20 to 60 continuous amino acids, 25 to 50 continuous amino acids, or 30 to 40 continuous amino acids. In an embodiment, the peptide linker (1) may consist of 30 amino acids. In addition, the peptide linker (1) may comprise at least one cysteine. Specifically, the peptide linker (1) may comprise one, two, or three cysteines. In addition, the peptide linker (1) may be derived from the hinge of an immunoglobulin. In an embodiment, the peptide linker (1) may be a peptide linker consisting of the amino acid sequence of SEQ ID NO: 3.

The peptide linker (2) may consist of 1 to 50 continuous amino acids, 3 to 30 continuous amino acids, or 5 to 15 continuous amino acids. In an embodiment, the peptide linker (2) may be (G4S)ₙ (where n is an integer of 1 to 10). Here, in (G4S)., n may be 1, 2, 3, 4, 5, 6, 7, 8, 9, or 10. In an embodiment, the peptide linker (2) may be a peptide linker consisting of the amino acid sequence of SEQ ID NO: 5.

In another aspect of the present invention, there is provided a pharmaceutical composition including, as an active ingredient, a dimer obtained by binding of two fusion proteins, each of which comprises an IL-2 protein and a CD80 protein, and a NK cell. The fusion protein comprising IL-2 or a variant thereof and CD80 or a fragment thereof is as described above.

Here, the binding between the fusion proteins constituting the dimer may be achieved by, but is not limited to, a disulfide bond formed by cysteines present in the linker. The fusion proteins constituting the dimer may be the same or different fusion proteins from each other. Preferably, the dimer may be a homodimer. An embodiment of the fusion protein constituting the dimer may be a protein having the amino acid sequence of SEQ ID NO: 9.

In the present invention, the cancer may be any one selected from the group consisting of gastric cancer, liver cancer, lung cancer, colorectal cancer, breast cancer, prostate cancer, ovarian cancer, pancreatic cancer, cervical cancer, thyroid cancer, larynx cancer, acute lymphoblastic leukemia, brain tumor, neuroblastoma, retinoblastoma, head and neck cancer, salivary cancer, and lymphoma.

A preferred dose of the pharmaceutical composition varies depending on the patient's condition and body weight, severity of disease, form of drug, route and duration of administration and may be appropriately selected by those skilled in the art. In the pharmaceutical composition for treating or preventing cancer of the present invention, the active ingredient may be comprised in any amount (effective amount) depending on application, use, dosage form, blending purpose, and the like, as long as the active ingredient can exhibit an anticancer activity. A conventional effective amount thereof will be determined within a range of 0.001% to 20.0% by weight, based on the total weight of the composition. Here, the term “effective amount” refers to an amount of an active ingredient capable of inducing an anticancer effect. Such an effective amount can be experimentally determined within the scope of common knowledge of those skilled in the art.

As used herein, the term “treatment” may be used to mean both therapeutic and prophylactic treatment. Here, prophylaxis may be used to mean that a pathological condition or disease of an individual is alleviated or relieved. In an embodiment, the term “treatment” includes both application or any form of administration for treating a disease in a mammal, including a human. In addition, the term includes inhibiting or slowing down a disease or disease progression; and includes meanings of restoring or repairing impaired or lost function so that a disease is partially or completely alleviated; stimulating inefficient processes; or alleviating a serious disease.

As used herein, the term “efficacy” refers to capability that can be determined by one or parameters, for example, survival or disease-free survival over a certain period of time such as one year, five years, or ten years. In addition, the parameter may include decrease of size of at least one tumor in an individual.

Pharmacokinetic parameters such as bioavailability and underlying parameters such as clearance rate may also affect efficacy. Thus, “enhanced efficacy” (for example, improvement in efficacy) may be due to enhanced pharmacokinetic parameters and improved efficacy, which may be measured by comparing clearance rate and tumor growth in laboratory animals or human subjects, or by comparing parameters such as survival, recurrence, or disease-free survival.

As used herein, the term “therapeutically effective amount” or “pharmaceutically effective amount” refers to an amount of a compound or composition effective to prevent or treat the disease in question, which is sufficient to treat the disease at a reasonable benefit/risk ratio applicable to medical treatment and does not cause adverse effects. A level of the effective amount may be determined depending on factors including the patient's health condition, kinds and severity of disease, activity of drug, the patient's sensitivity to drug, mode of administration, time of administration, route of administration and excretion rate, duration of treatment, formulation or simultaneously used drugs, and other factors well known in the medical field. In an embodiment, the therapeutically effective amount means an amount of drug effective to treat cancer.

Here, the pharmaceutical composition may further include a pharmaceutically acceptable carrier. The pharmaceutically acceptable carrier may be any carrier as long as the carrier is a non-toxic substance suitable for delivery to a patient. Distilled water, alcohol, fat, wax, and inert solid may be contained as the carrier. A pharmaceutically acceptable adjuvant (buffer, dispersant) may also be contained in the pharmaceutical composition.

Specifically, by including a pharmaceutically acceptable carrier in addition to the active ingredient, the pharmaceutical composition may be prepared into a parenteral formulation depending on its route of administration using conventional methods known in the art. Here, the term “pharmaceutically acceptable” means that the carrier does not have more toxicity than the subject to be applied (prescribed) can adapt while not inhibiting activity of the active ingredient.

When the pharmaceutical composition is prepared into a parenteral formulation, it may be made into preparations in the form of injections, transdermal patches, nasal inhalants, or suppositories with suitable carriers according to methods known in the art. In a case of being made into injections, sterile water, ethanol, polyol such as glycerol or propylene glycol, or a mixture thereof may be used as a suitable carrier; and an isotonic solution, such as Ringer's solution, phosphate buffered saline (PBS) containing triethanol amine or sterile water for injection, and 5% dextrose, or the like may preferably be used. Formulation of pharmaceutical compositions is known in the art, and reference may specifically be made to Remington's Pharmaceutical Sciences (19^{th} ed., 1995) and the like. This document is considered part of the present description.

A preferred dose of a dimer in the pharmaceutical composition may range from 0.01 μg/kg to 10 g/kg, or 0.01 mg/kg to 1 g/kg, per day, depending on the patient's condition, body weight, sex, age, severity of the patient, and route of administration. The dose may be administered once a day or may be divided into several times a day. Such a dose should not be construed as limiting the scope of the present invention in any aspect. In addition, NK cells in the pharmaceutical composition may be administered at an amount of 1×10² to 1×10¹³ cells, 1×10⁷ to 1.5×10¹¹ cells, with being adjusted appropriately in the range showing a pharmacological effect.

Subjects to which the pharmaceutical composition can be applied (prescribed) are mammals and humans, with humans being particularly preferred. In addition to the active ingredient, the pharmaceutical composition of the present application may further include any compound or natural extract, which has already been validated for safety and is known to have anticancer activity so as to boost or reinforce anticancer activity.

In still yet another aspect of the present invention, there is provided a use of a fusion protein dimer comprising an IL-2 protein and a CD80 protein, and a NK cell for treating cancer.

In still yet another aspect of the present invention, there is provided a use of a fusion protein dimer comprising an IL-2 protein and a CD80 protein, and NK cells for enhancing a therapeutic effect on cancer.

In still yet another aspect of the present invention, there is provided a use of a fusion protein dimer comprising an IL-2 protein and a CD80 protein, and NK cells for manufacture of a medicament for treating cancer.

In still yet another aspect of the present invention, there is provided a method for treating cancer and/or a method for enhancing a therapeutic effect on cancer, including administering, to a subject, a fusion protein comprising an IL-2 protein and a CD80 protein or a fusion protein dimer where the two fusion proteins are linked, and a NK cell.

Here, the fusion protein dimer and the NK cells may be administered simultaneously or sequentially. In this case, the order of administration may be determined such that the administration of the fusion protein dimer may be followed by the administration of NK cells, or the administration of the NK cells may be followed by the fusion protein dimer.

The subject may be an individual suffering from cancer or an infectious disease. In addition, the subject may be a mammal, preferably a human. The fusion protein comprising an IL-2 protein and a CD80 protein, or the fusion protein dimer where the two fusion proteins are linked is as described above.

Route of administration, dose, and frequency of administration of the fusion protein or fusion protein dimer and NK cells may vary depending on the patient's condition and the presence or absence of side effects, and thus the fusion protein or fusion protein dimer may be administered to a subject in various ways and amounts. The optimal administration method, dose, and frequency of administration can be selected in an appropriate range by those skilled in the art. In addition, the fusion protein or fusion protein dimer may be administered in combination with other drugs or physiologically active substances whose therapeutic effect is known with respect to a disease to be treated, or may be formulated in the form of combination preparations with other drugs.

Due to IL-2 activity, the fusion protein in an embodiment of the present invention can activate immune cells such as natural killer cells. Thus, the fusion protein can be effectively used for cancer and infectious diseases. In particular, it was identified that as compared with the wild type, an IL-2 variant with two to five amino acid substitutions, in particular, an IL-2 variant that comprises amino acid substitutions at two, three, four, or five positions among the positions selected from the group consisting of R38A, F42A, Y45A, E61R, and L72G, has low binding ability for the IL-2 receptor alpha chain and thus exhibits improved characteristics with respect to pharmacological side effects of conventional IL-2. Thus, such an IL-2 variant, when used alone or in the form of a fusion protein, can decrease incidence of vascular (or capillary) leakage syndrome (VLS), a problem with IL-2 conventionally known.

### MODE FOR THE INVENTION

Hereinafter, the present invention will be described in more detail by way of the following examples. However, the following examples are only for illustrating the present invention, and the scope of the present invention is not limited thereto.

### I. Preparation of Fusion Protein Dimer Comprising IL-2 and CD80

### Preparatory Example 1. Preparation of a hCD80-Fc-IL-2 Variant (2M): GI-101

In order to produce a fusion protein comprising a human CD80 fragment, a Fc domain, and an IL-2 variant, a polynucleotide including a nucleotide sequence (SEQ ID NO: 8) encoding a fusion protein comprising a signal peptide (SEQ ID NO: 1), a CD80 fragment (SEQ ID NO: 2), a linker-bound Ig hinge (SEQ ID NO: 3), a Fc domain (SEQ ID NO: 4), a linker (SEQ ID NO: 5), and an IL-2 variant (2M) in which two amino acids are substituted (R38A, F42A) (SEQ ID NO: 6) in this order from N-terminus was synthesized through Invitrogen GeneArt Gene Synthesis service of ThermoFisher Scientific Inc., and cloned into a pcDNA3_4 vector. In addition, the vector was introduced into CHO cells (EXPI-CHO™) to express a fusion protein of SEQ ID NO: 9. After introducing the vector, the CHO cells were cultured in an environment of 37° C., 125 RPM, and 8% CO₂ for 7 days, and then collected to purify a fusion protein. The purified fusion protein dimer was named as “GI-101.”

Purification was performed using chromatography including MabSelect SuRe protein A resin. The fusion protein was bound under the condition of 25 mM Tris, 25 mM NaCl, and pH 7.4. Then, it was eluted with 100 mM NaCl and 100 mM acetic acid at pH 3. After putting 20% of 1M Tris-HCl at pH 9 into a collection tube, the fusion protein was collected. The collected fusion protein was dialyzed into PBS buffer for 16 hours to change.

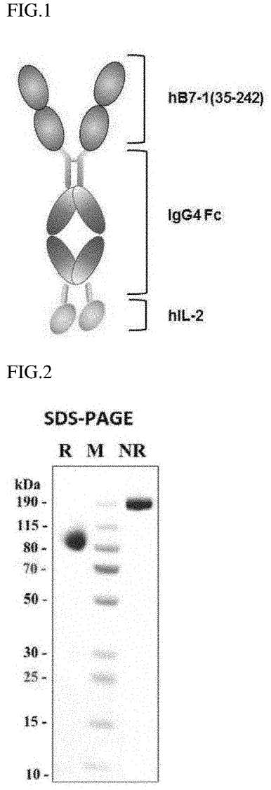

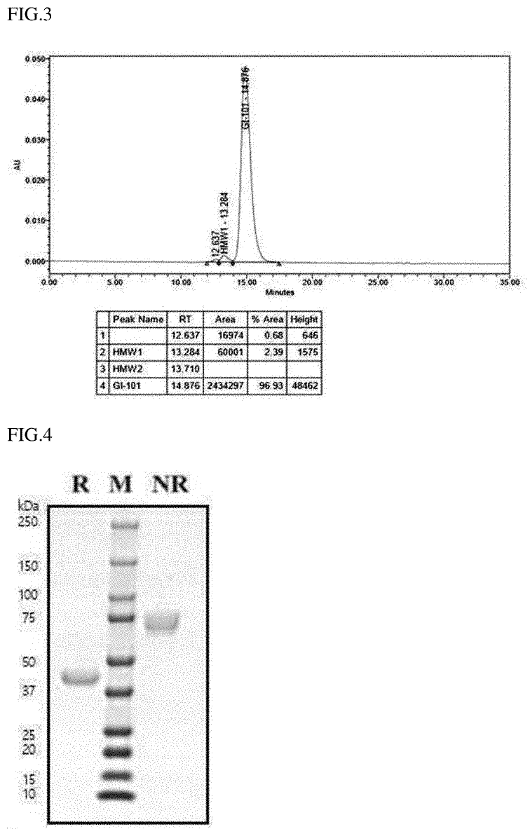

Then, absorbance at a wavelength of 280 nm over time was measured by using size exclusion chromatography with TSKgel G3000SWXL column (TOSOH Bioscience) to obtain a high concentration of fusion protein. At this time, the isolated and purified fusion protein was subjected to SDS-PAGE under the reducing (R) or non-reducing (NR) conditions, and stained with Coomassie blue to confirm its purity (FIG. 2). It was confirmed that the fusion protein was included at a concentration of 2.78 mg/mL as detected using NanoDrop. Also, the result analyzed using size exclusion chromatography is as shown in FIG. 3.

### Preparatory Example 2. Preparation of a Fc-IL-2 Variant (2M) Dimer: Fc-IL-2v2

In order to produce a fusion protein comprising a Fc domain and an IL-2 variant, a polynucleotide including a nucleotide sequence (SEQ ID NO: 45) encoding a fusion protein comprising a signal peptide (SEQ ID NO: 1), Ig hinge (SEQ ID NO: 38), a Fc domain (SEQ ID NO: 4), a linker (SEQ ID NO: 5), and an IL-2 variant (2M) in which two amino acids are substituted (R38A, F42A) (SEQ ID NO: 6) in this order from N-terminus was synthesized through Invitrogen GeneArt Gene Synthesis service of ThermoFisher Scientific Inc., and cloned into a pcDNA3_4 vector. In addition, the vector was introduced into CHO cells (EXPI-CHO™) to express a fusion protein of SEQ ID NO: 44. After introducing the vector, the CHO cells were cultured in an environment of 37° C., 125 RPM, and 8% CO₂ for 7 days, and then collected to purify a fusion protein dimer. The purified fusion protein dimer was named as “Fc-IL2v2.”

The purification and collection of the fusion protein were performed in the same manner as in the Preparatory Example 1. The isolated and purified fusion protein was subjected to SDS-PAGE under the reducing (R) or non-reducing (NR) conditions, and stained with Coomassie blue to confirm its purity (FIG. 4). As a result, it was confirmed that the fusion protein forms a dimer. Also, the result analyzed using size exclusion chromatography is as shown in FIG. 5.

### Preparatory Example 3. Preparation of a hCD80-Fc Dimer: hCD80-Fc

In order to produce a fusion protein comprising a human CD80 fragment and a Fc domain, a polynucleotide (SEQ ID NO: 39) including a nucleotide sequence encoding a fusion protein comprising a signal peptide (SEQ ID NO: 1), a CD80 fragment (SEQ ID NO: 2), a linker-bound Ig hinge (SEQ ID NO: 3), and a Fc domain (SEQ ID NO: 4) in this order from N-terminus was synthesized through Invitrogen GeneArt Gene Synthesis service of ThermoFisher Scientific Inc., and cloned into a pcDNA3_4 vector. In addition, the vector was introduced into CHO cells (EXPI-CHO™) to express a fusion protein of SEQ ID NO: 40. After introducing the vector, the CHO cells were cultured in an environment of 37° C., 125 RPM, and 8% CO₂ for 7 days, and then collected to purify a fusion protein dimer. The purified fusion protein dimer was named “hCD80-Fc.”

Purification was performed using chromatography including MabSelect SuRe protein A resin. The fusion protein was bound under the condition of 25 mM Tris, 25 mM NaCl, and pH 7.4. Then, it was eluted with 100 mM NaCl and 100 mM acetic acid at pH 3. After putting 20% of 1 M Tris-HCl at pH 9 into a collection tube, the fusion protein was collected. The collected fusion protein was dialyzed into PBS buffer for 16 hours to change.

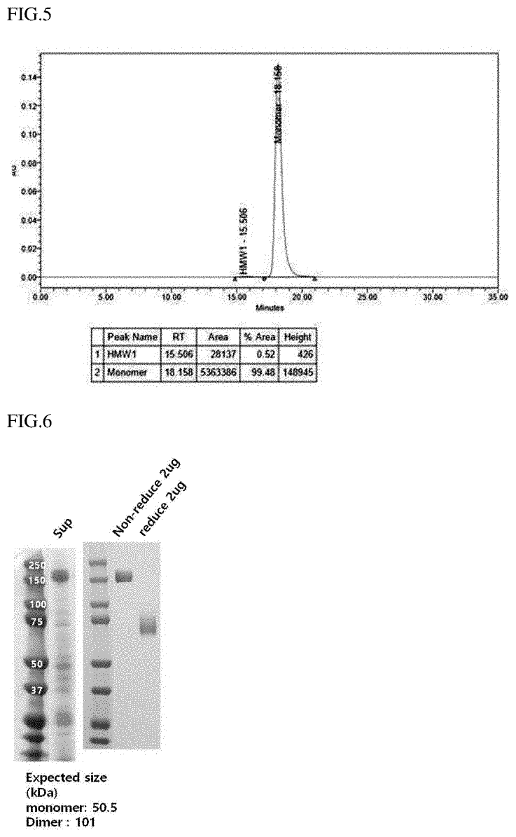

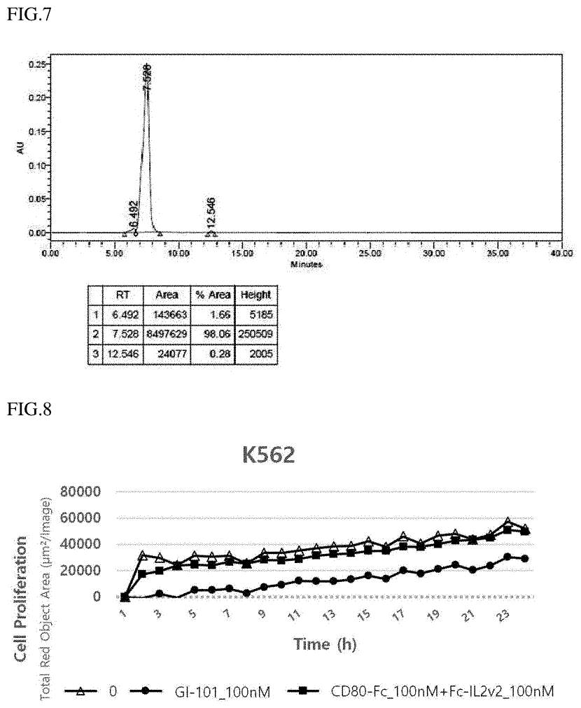

Then, absorbance at a wavelength of 280 nm over time was measured by using size exclusion chromatography with TSKgel G3000SWXL column (TOSOH Bioscience) to obtain a high concentration of fusion protein. At this time, the isolated and purified fusion protein was subjected to SDS-PAGE under the reducing (R) or non-reducing (NR) conditions, and stained with Coomassie blue to confirm its purity (FIG. 6). As a result, it was confirmed that the fusion protein forms a dimer. Also, the result analyzed using size exclusion chromatography is as shown in FIG. 7.

### Preparatory Example 4. Preparation of mCD80-Fc-IL-2 Variant (2M): mGI101

In accordance with the same method as in Preparatory Example 1, a mouse type GI-101 comprising a mouse-derived CD80 and IL-2 was manufactured. Specifically, in order to produce a fusion protein comprising a mouse CD80, an Fc domain, and an IL-2 variant, a polynucleotide was synthesized through the Invitrogen GeneArt Gene Synthesis service of ThermoFisher Scientific. The polynucleotide comprises a nucleotide sequence (SEQ ID NO: 14) which encodes a fusion protein that comprises a signal peptide (SEQ ID NO: 1), a mCD80 (SEQ ID NO: 13), a linker-bound Ig hinge (SEQ ID NO: 3), an Fc domain (SEQ ID NO: 4), a linker (SEQ ID NO: 5), and an IL-2 variant (2M) (R38A, F42A) (SEQ ID NO: 6) with two amino acid substitutions, in this order, from the N-terminus. The polynucleotide was inserted into pcDNA3_4 vector. In addition, the vector was introduced into CHO cells (EXPI-CHO™) to express the fusion protein of SEQ ID NO: 47. After the vector was introduced, culture was performed for 7 days in an environment of 37° C., 125 RPM, and 8% CO₂. Then, the culture was harvested and the fusion protein was purified therefrom. The purified fusion protein was designated “mGI101”.

### II. Preparation and Culture of NK Cells

### Preparation Example 1. Isolation of Peripheral Blood Mononuclear Cells (PBMC) Derived CD3(−)CD56(+) Natural Killer Cells

In order to obtain CD3(−) cells, the number of PBMCs (peripheral blood mononuclear cells, Zen-Bio. Inc, NC 27709, USA, Cat #: SER-PBMC-200-F) was measured using an ADAM-MC2 automated cell counter (NanoEnTek, purchased from Cosmo Genetech Co., Ltd.). The PBMCs were transferred to a new tube, and then centrifuged at 300×g for 5 minutes at a temperature of 4° C. 0.5% (v/v) of bovine serum albumin (BSA) and 2 mM of EDTA were included in PBS to prepare MACS buffer (pH 7.2). After centrifugation was completed, a cell pellet was treated with 80 μl of MACs buffer and 20 μl of CD3 magnetic beads (Miltenyi biotech, 130-050-101) per 1×10⁷ cells to suspend, and then reacted at a temperature of 4° C. for 15 minutes. 10 mL of MACs buffer was added for washing and centrifuged at 300×g for 10 minutes at a temperature of 4° C., and then the cell pellet was resuspended in 0.5 mL of MACs buffer.

2 mL of MACs buffer was first flowed into the LD column (Miltenyi Biotec, Bergisch Gladbach, Germany, Cat #: 130-042-901), and then the cell suspension was flowed. Then, CD3(−) cells passing through the LD column were obtained. At this time, CD3(−) cells were obtained by flowing 2 mL of MACs buffer three times so that the cells remaining in the LD column could be sufficiently separated. The obtained CD3(−) cells were counted using a cell counter, and then placed in a new tube and centrifuged at 300×g for 5 minutes at a temperature of 4° C. Then, the supernatant was removed, and then 80 μl of MACs buffer and 20 μl of CD56 magnetic beads (Miltenyi biotech, Cat #: 130-050-401) were added per 1×10⁷ cells, followed by reaction at a temperature of 4° C. for 15 minutes. 10 mL of MACs buffer was added for washing and centrifuged at 300×g for 10 minutes at a temperature of 4° C., and then the cell pellet was resuspended in 0.5 mL of MACs buffer.

3 mL of MACs buffer was first flowed into the LS column (Miltenyi Biotec, Bergisch Gladbach, Germany, Cat #: 130-042-901), and then the cell suspension was flowed. At this time, 2 mL of MACs buffer was flowed three times so that the cells remaining in the LS column could be sufficiently separated. Then, after the LS column was separated from a magnet stand, 5 mL of MACs buffer was added, and pressure was applied with a piston to obtain CD3(−)CD56(+) natural killer cells. The obtained CD3(−)CD56(+) natural killer cells was placed in a new tube and centrifuged at 300×g for 5 minutes at a temperature of 4° C. After removing the supernatant, the cells were suspended in the culture media, and the number of suspended cells was measured using a cell counter.

### Preparation Example 2. Culture and Obtainment of CD3-CD56+ Natural Killer Cells

100 μl of CD335 (NKp46)-biotin and 100 μl of CD2-biotin included in a NK Cell Activation/Expansion Kit (Cat #: 130-112-968)(Miltenyi Biotec, Bergisch Gladbach, Germany) were placed in a 1.5 mL microtube and mixed. 500 μl of Anti-Biotin MACSiBead Particles was added and mixed. Then, 300 μl of MACs buffer was added, and mixed at 2° C. to 8° C. for 2 hours using a microtube rotator.

Then, 5 μl of NK activation beads per 1×10⁶ cells was transferred to a new tube. 1 mL of PBS was added and centrifuged at 300×g for 5 minutes. After removing the supernatant, an RPMI1640 medium added with 5% human AB serum (Cat #:H4522)(Sigma, St. Louis, Mo., US) to be used was added on the basis of μl per 10⁶ NK cells, and suspended beads, followed by inoculating into the CD3-CD56+ natural killer cells isolated in Preparation Example 1.

Next, the CD3-CD56+ natural killer cells were seeded in a 24-well plate, and an RPMI1640 medium added with 5% human AB serum (Cat #:H4522)(Sigma, St. Louis, Mo., US) and rhIL-2(500 IU/mL) was added thereto, followed by culturing under the condition of 37° C. and 5% CO₂. Then, the number of cells was determined every 2 days to subculture in the order of a 12-well plate, a 6-well plate, and a 25T flask when the cells occupied 80% or more of culture vessel (confluency), and finally all cells were harvested on day 21.

### Preparation Example 3. Culture and Collection of Mouse-Derived Natural Killer Cell

### Preparation Example 3.1. Preparation of Mouse Spleen Cell and Bone Marrow

To obtain mouse-derived natural killer cells, first mouse spleen cells and bone marrow were prepared. Specifically, the spleen and femur were extracted from a 6-week-old female Balb/c (ORIENT BIO Inc.), and the fat and muscle were removed as much as possible while taking care of the femur not to break. The extracted femur was placed in 70% ethanol, followed by in a 50 mL tube containing 5 mL of PBS, and the spleen were immediately transferred to a 50 mL tube containing 5 mL of PBS, and then stored on ice. A 70 μm strainer was overlapped a 50 mL tube containing 5 mL of FACS buffer and prepared for the spleen and bone marrow, respectively. The composition of FACS buffer A is as described in table 1 below.

TABLE 1

| Composition | | | | | Final |
|---|---|---|---|---|---|
| Product | | Manufacturer | Volume | | concentration |
| FACS | FBS | Hyclone | 15 | mL | 3% |
| buffer | EDTA | Welgene | 10 | mL | 10 mM |
| A | HEPES | Welgene | 10 | mL | 20 mM |
| | PolymyxinB | Merk | 300 | mL | 10 g/mL |
| | Penicillin/ | Biolegend | 5 | mL | 10,000 U/mL |
| | Streptomycin | | | | penicillin/ |
| | | | | | 10,000 g/mL |
| | | | | | Streptomycin |
| | 100 mM | Gibco | 5 | mL | 1 mM |
| | sodium | | | | |
| | pyruvate | | | | |
| | PBS | Sigma | to 500 | mL | |

After the tissue was mashed with a syringe stick, 5 mL of FACS buffer A was added to collect the cells. Then, centrifugation was performed at 1,300 rpm for 5 minutes at 4° C., and the supernatant was removed. The spleen was dissolved with 3 mL of ACK lysis buffer to remove red blood cells, and then incubated on ice for 3 minutes. After 3 minutes, FACS buffer A was added to make the total volume of 20 mL. The resultant was vortexed, and then centrifuged at 1,300 rpm for 5 minutes at 4° C. and the supernatant was removed. Red blood cells were removed by repeating the above process until the color of a pellet turned pink, and dissolved with 10 mL of FACS buffer A to count the cells.

Both sides of the femur bone on the 70 μm strainer for bone marrow was cut, and FACS buffer A was flowed into holes of the bone using a 1 ml needle of a syringe filled with 10 mL of FACS buffer A while moving back and forth, and then the cells were collected while flowing FACS buffer A to the cut tissue as well. The cells were collected, and then centrifuged at 1,300 rpm for 5 minutes at 4° C. Then, the supernatant was removed and dissolved with 10 mL of FACS buffer A, followed by counting the cell.

### Preparation Example 3.2. Isolation and Culture of Mouse NK Cell

Using an NK cell isolation Kit (#130-115-818), NK cells were isolated from the spleen and bone marrow which were isolated from the 6-week-old female Balb/c (ORIENT BIO Inc.) mouse in Preparation Example 3.1 above. After isolation, centrifugation was performed at 1,300×g at 4° C., and the supernatant was removed.

40 μl of MACS buffer per 10⁷ cells (600 μl for spleen, 200 μl for bone marrow) was added to resuspend the cell pellet, and 40 μl of NK cell cocktail per 10⁷ cells (150 μl for spleen, 50 μl for bone marrow) was added. However, in this case, more than 5×10⁷ cells showed aggregation phenomenon, so they were divided and mixed. Then, centrifugation was performed at 300×g at 4° C., and the supernatant was removed.

2 mL of washing buffer per 10⁷ cells was added (30 mL for spleen, 10 mL for bone marrow), washed, centrifuged at 4° C. at 300×g, and the supernatant was removed. Then, 80 μl of MACS buffer per 10⁷ cells was added (1.2 mL for spleen, 400 μl for bone marrow), and then 20 μl of anti-biotin microbeads per 10⁷ cells was added (300 μl for spleen, 100 μl for bone marrow), mixed, and cultured in a refrigerator for 10 minutes.

500 μl of cells mixed with anti-biotin microbeads was flowed into an MS column to obtain a supernatant that passed through the column. The same process was repeated to obtain the supernatant that passed through the column, and centrifuged at 4° C. at 300×g. Then, the supernatant was removed. The cells were counted by resuspending in a GC-RPMI medium (1×) (spleen: 6.95×10⁵/mL viability 59%, bone marrow: 1.58×10⁶/mL viability 64%). The composition of a GC-RPMI medium is as described in Table 2 below.

TABLE 2

| | | | | Final |
|---|---|---|---|---|
| Composition | | | | con- |
| Product | | Manufacturer | Cat.# | centration |
| GC- | RPMI 1640 | Welgene | LM 011-01 | |
| RPMI | Pen-Strep | Welgene | LS 202-02 | 1× |
| | Gentamicin | Gibco | 15750-060 | 50 μg/mL |
| | Sodium pyruvate | Welgene | LS 013-01 | 1 mM |
| | 2-Mercaptoethanol | Gibco | 21985-023 | 55 μM |
| | NEAA | Gibco | 11140050 | 2 mM |
| | L-Glutamine | Gibco | 25030149 | 2 mM |
| | FBS | Hyclone | SH30084.03 | 10% |

After seeding 3.5×10⁵ NK cells isolated from the spleen and bone marrow in a 60 mm culture vessel, 50 ng/mL of rmIL-2 was treated and cultured for 14 days.

### III. Preparation of Cancer Cells and Construction of Mouse Model

### Preparation Example 4. Preparation of Human-Derived Carcinoma Cell Line and Culture Medium Thereof

A culture solution suitable for each cancer cell line was used with reference to Table 3 below.

TABLE 3

| Cancer cell | | | | Components of |
|---|---|---|---|---|
| line | Organism of origin | Disease | Manufacturer | culture medium |
| K562 | Homo sapiens, Human | chronic myeloid | ATCC | RPMI 1640 + 10% |
| | | leukemia | | FBS |
| MDA-MB- | Homo sapiens, Human | breast cancer | Korean | High glucose |
| 231 | | | Cell Line | RPMI 1640 + 10% |
| | | | Bank | FBS |
| HCT-116 | Homo sapiens, Human | colon cancer | ATCC | McCoy's 5A + 10% |
| | | | | FBS |
| A549 | Homo sapiens, Human | lung cancer | ATCC | RPMI 1640 + 10% |
| | | | | FBS |

Specifically, 2×10⁶ cells of cancer cell lines were resuspended in 8 mL of each culture solution and cultured in a 25T flask. When recover the cells, 1 mL of trypsin-EDTA (0.25%) was treated and then reacted in 5% CO₂ for 2 minutes for adherent type cells. Then, 5 mL of culture solution was added to recover the cells that had detached from the flask, and centrifuged at 300×g for 5 minutes.

Table 4 below shows specific culture solution compositions for each cancer cell lines.

TABLE 4

| Composition | | | | | | | Final |
|---|---|---|---|---|---|---|---|
| Product | | | Manufacturer | Cat. # | Volume | | conc. |
| RPMI1640 | Basic | RPMI 1640 | Welgene | LM011-01 | 500 | mL | |
| (10% | components | Medium(1×), | | | | | |
| FBS) | | liquid | | | | | |
| | | FBS | HYCLONE ™ | SV30207.02 | 50 | mL | 10% |
| | | Penicillin- | Welgene | LS 202-02 | 0.5 | mL | 1× |
| | | Streptomycin | | | | | |
| | | Solutions(×100) | | | | | |
| High | Basic | High-glucose | ATCC | 30-2001 | 500 | mL | |
| glucose | components | RPMI 1640 | | | | | |
| RPMI1640 | | Medium | | | | | |
| (10% | | FBS | HYCLONE ™ | SV30207.02 | 50 | mL | 10% |
| FBS) | | Penicillin- | Welgene | LS 202-02 | 0.5 | mL | 1× |
| | | Streptomycin | | | | | |
| | | Solutions(×100) | | | | | |
| McCoy's | Basic | McCoy's | ATCC | 30-2007 | 500 | mL | |
| (10% | components | 5A(ATCC) | | | | | |
| FBS) | | FBS | HYCLONE ™ | SV30207.02 | 50 | mL | 10% |
| | | Penicillin- | Welgene | LS 202-02 | 0.5 | mL | 1× |
| | | Streptomycin | | | | | |
| | | Solutions(×100) | | | | | |

### Preparation Example 5. Preparation of Mouse-Derived Carcinoma Cell Line and Culture Medium Thereof

CT26.WT, a Mus musculus colon carcinoma cell, was purchased from ATCC (American Type Culture Collection, USA) (Table 5). Carcinoma cells to be used in the experiment were thawed, placed in a flask for cell culture, and cultured at 37° C., in a 5% CO₂ incubator (MCO-170M, Panasonic, Japan). The cells cultured on the day of cell line transplantation were placed in a centrifuge tube and collected, and then centrifuged at 125×g for 5 minutes to remove the supernatant. Then, PBS was added to prepare cell suspension (5×10⁷ cells/mL), dispensed in aliquot for 9 mice, and stored on ice until administration.

TABLE 5

| | Organism of | | | Final |
|---|---|---|---|---|
| | origin | Disease | Manufacturer | concentration |
| CT26 | Mus musculus, | colon | ATCC | 5 × 10⁵ |
| | mouse | carcinoma | | |

Fetal bovine serum (FBS; 16000-044, Thermofisher scientific, USA), penicillin-streptomycin (10,000 units/mL of penicillin and 10,000 μg/mL of streptomycin; 15140122, Thermofisher scientific, USA) and RPMI1640(A1049101, Thermofisher scientific, USA) were mixed to have the composition described in Table 6 below per 100 mL and used as a culture medium for carcinoma cells.

TABLE 6

| Composition | | | | | | Final |
|---|---|---|---|---|---|---|
| Product | | Manufacturer | Cat.# | Volume | | concentration |
| Culture | FBS | Thermofisher | 16000-044 | 10 | mL | 10% |
| medium | | scientific | | | | |
| for | Penicillin & | Thermofisher | 15140122 | 1 | mL | 10,000 U/mL |
| cancer | Streptomycin | scientific | | | | penicillin |
| cells | | | | | | & 10,000 μg/mL |
| | | | | | | Streptomycin |
| | RPMI 1640 | Thermofisher | A1049101 | To 100 | mL | |
| | | scientific | | | | |

### Preparation Example 6. Preparation of Carcinoma Mouse Model Preparation Example 6.1. Quarantine and Acclimation Processes for Experimental Mouse

36 female 12-week-old BALB/c mice were purchased from ORIENT BIG Inc. Mice were brought into the animal lab and acclimated for 5 days prior to being used in the experiment. When received a mouse, they were evaluated for appearance and weighted to measure body weight. General symptoms were observed once a day during the acclimatization period for 5 days, and the body weight was measured at the end of the acclimation period, and then general symptoms and body weight changes were checked to evaluate the health condition of the mice. Mice with abnormality were euthanized under CO₂ gas anesthesia.

Information about laboratory mice is summarized and shown in Table 7 below.

TABLE 7

| | Strain of origin | Purchased from | Age | Gender |
|---|---|---|---|---|
| Mouse | Balb/c | OrientBio | 12 weeks old | female |

### Preparation Example 6.2. Transplantation of Carcinoma Cell Line

For a tumor growth inhibition model, the body weight was measured the next day after the end of the quarantine and acclimatization period, and then the CT26 cell suspension (5×10⁵ cells/0.1 mL) prepared for healthy animals was dispensed, filled in a disposable syringe, and administered subcutaneously (0.1 mL/head) to the right back of the mouse to transplant. General symptoms were observed once a day during the engraftment and growth period following cell line transplantation.

### Preparation Example 6.3. Grouping of Tumor Growth Inhibition Mouse Models

After a certain period of CT26 cell transplantation, the tumor volume and body weight were measured for mice without abnormal health status, and were divided into 4 groups (9 mice per group) so that the average of each group reached 50 mm³.

### IV. Determination of Anticancer Activity of NK Cell and Fusion Protein Dimer: In Vitro

### Example 1. Determination of Cancer Cell Growth Inhibitory Effect of NK Cell and/or Fusion Protein Dimer Against Various Carcinoma

Referring to a plate design to be used for a 96-well plate, 50 μl of 0.01% poly-L-ornithine solution (Cat #. P4957)(Sigma Aldrich, US) was respectively dispensed into well and coated. Then, it was left at room temperature for 1 hour. After 1 hour, the dispensed 0.01% poly-L-ornithine solution was removed and completely dried at room temperature for 1 hour. 2 μl of CELLTRACKER™ Deep Red Dye (Cat #C34565)(Thermo Scientific, Waltham, Mass., USA) was added to the cancer cells (target cell) prepared at 4×10⁵ cells/mL and allowed to react for 60 minutes at 37° C. and 5% CO₂ condition.

After the reaction, the resultant was centrifuged at 300×g for 5 minutes. The supernatant was removed, and then dissolved in RPMI1640+5% hABS culture solution to 4×10⁵ cells/mL. 50 μl of the prepared cancer cells was dispensed into a well in a coated 96-well plate, respectively. Then, the prepared plate was placed in an INCUCYTE® Live-Cell Analysis system (Satorius, Germany) instrument, and then allowed to stabilize for 10 minutes. A culture solution containing a test material was prepared in the RPMI1640+5% hABS referring to Table 8 below.

TABLE 8

| Test substance | control | GI-101 | CD80-Fc + Fc-IL2v2 |
|---|---|---|---|
| Concentration | 0 nM | 100 nM | CD80-Fc: 100 nM, Fc-IL2v2: |
| | | | 100 nM |

Natural killer cells (effector cells) were prepared by suspending in RPMI1640+5% hABS culture solution to 4×10⁵ cells/mL. Referring to table 9 below, the natural killer cells prepared in Preparation examples 1 and 2 were added to each well in the plate having dispensed cancer cells, and then 100 μl of culture solution treated with INCUCYTE® CytoTox (250 nM) was added.

TABLE 9

| | E/T ratio | | | | |
|---|---|---|---|---|---|
| | 0/1 | 1/3 | 1/1 | 3/1 | 10/1 |
| Number of NK | — | 6.7 × 10³ | 2 × 10⁴ | 6 × 10⁴ | 2 × 10⁵ |
| cells | | | | | |
| Number of | 2 × 10⁴ | 2 × 10⁴ | 2 × 10⁴ | 2 × 10⁴ | 2 × 10⁴ |
| cancer cells | | | | | |

Then, it was placed into the INCUCYTE® Live-Cell Analysis system and analyzed for 3 days with time interval of 30 minutes.

### Example 1.1. Determination of Efficacy of Fusion Protein Dimer Alone in the Presence of Target Cancer Cell without NK Cell

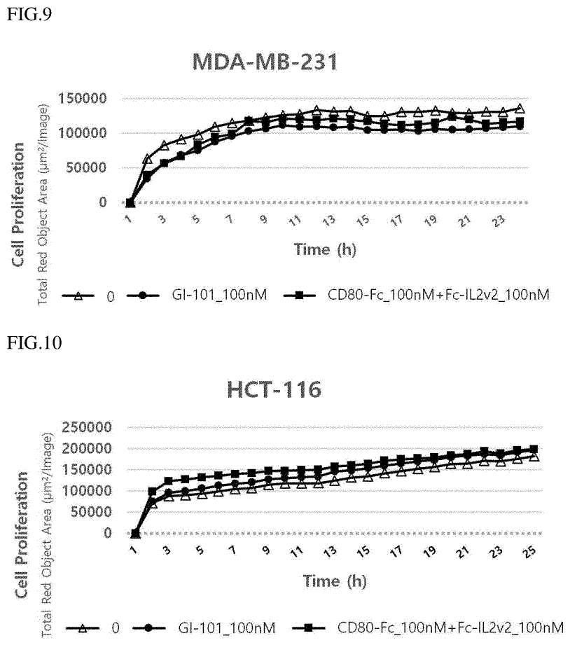

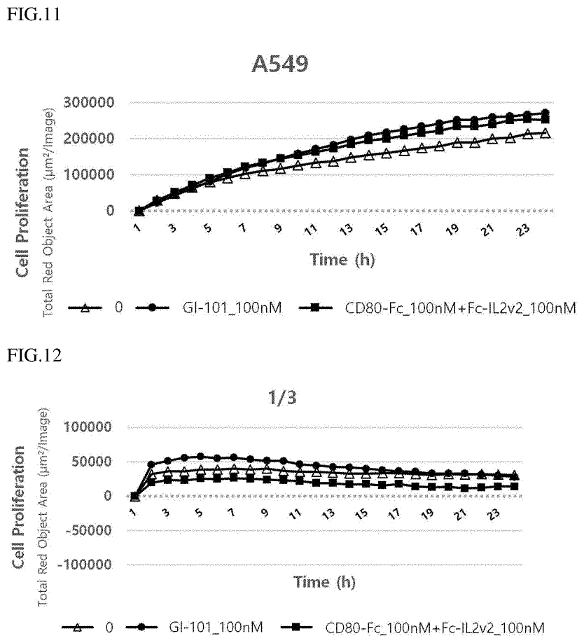

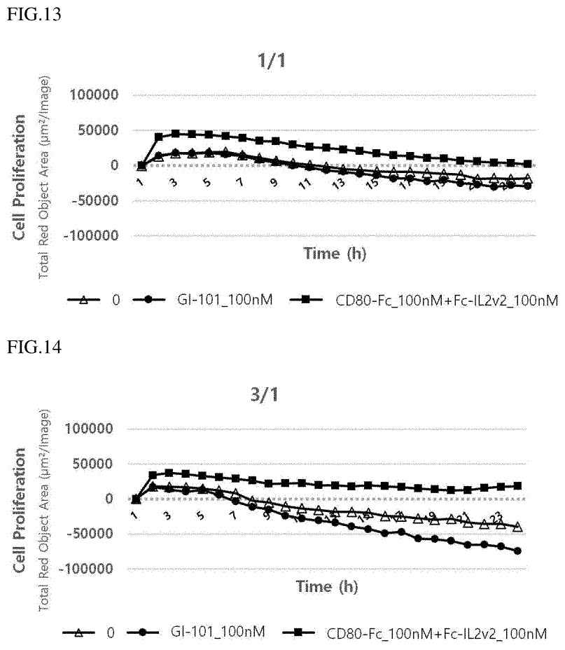

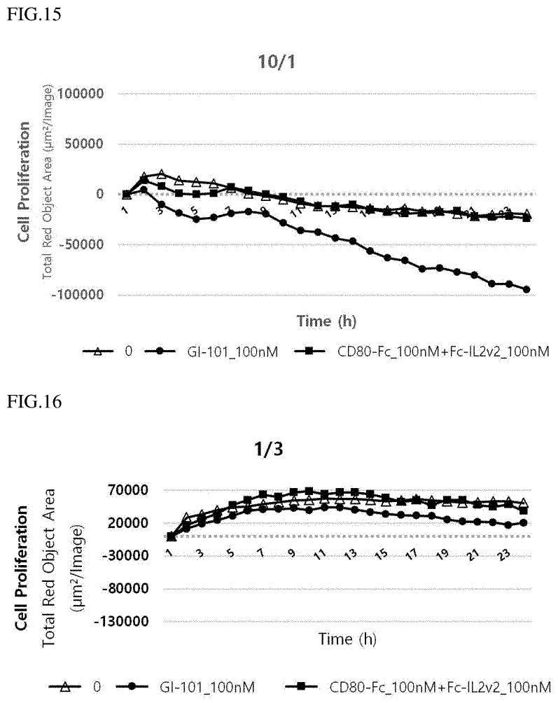

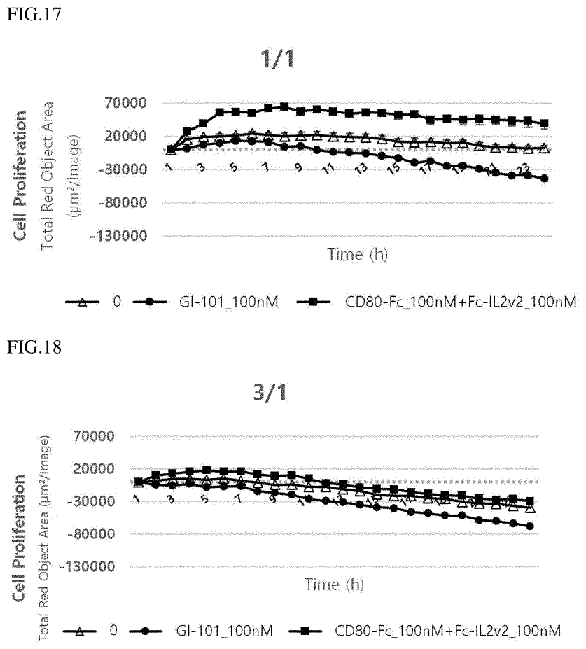

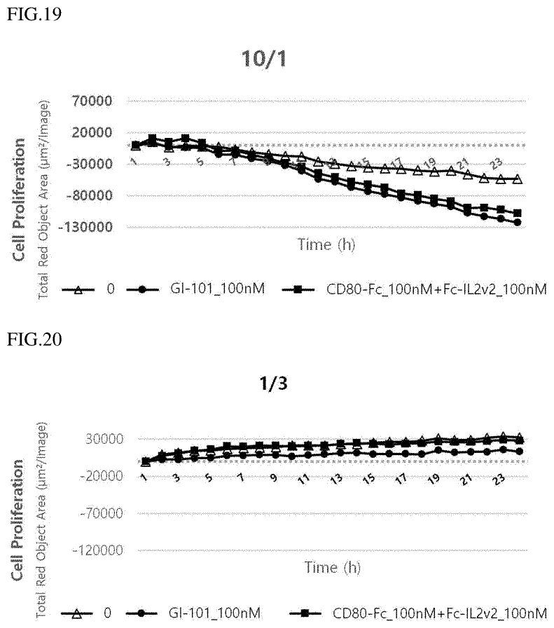

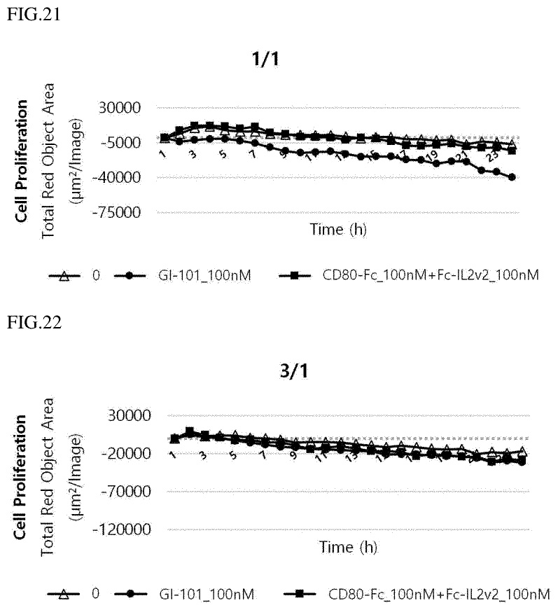

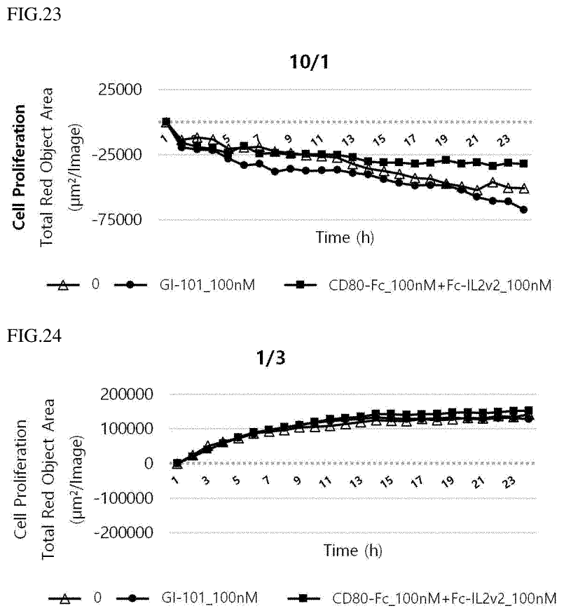

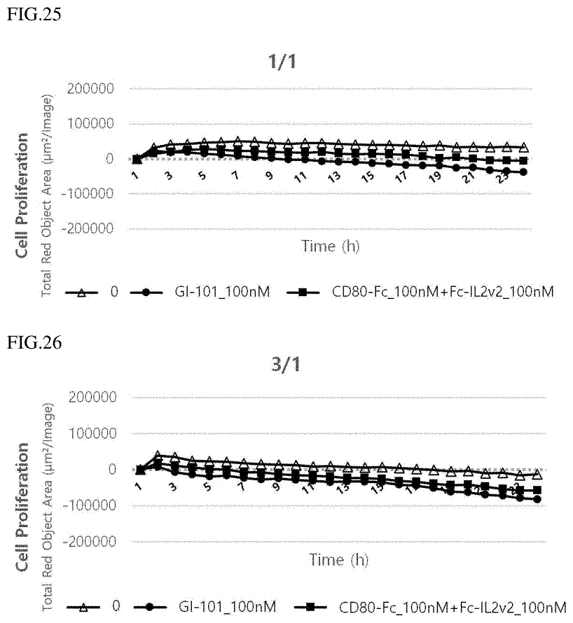

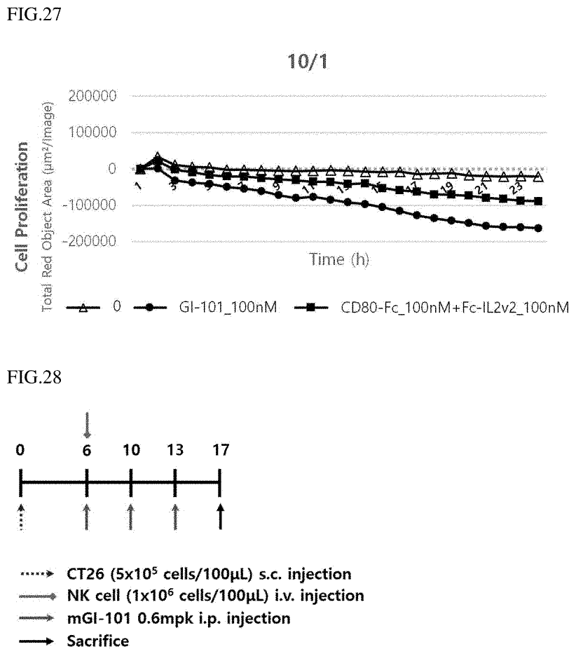

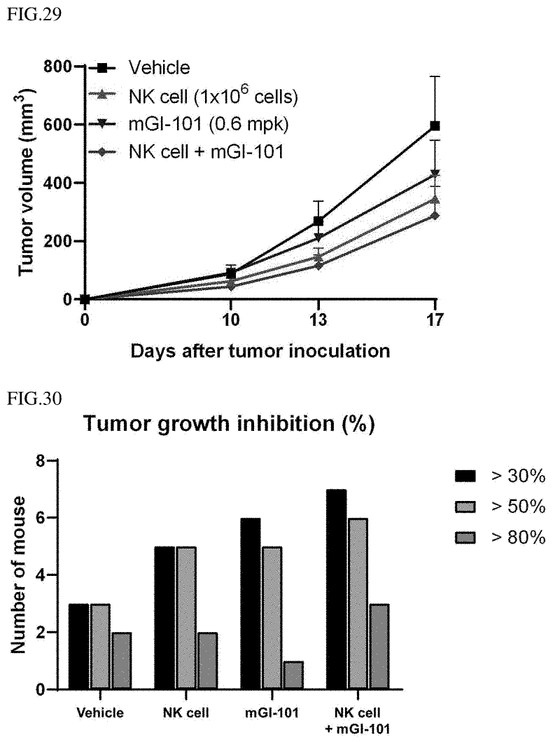

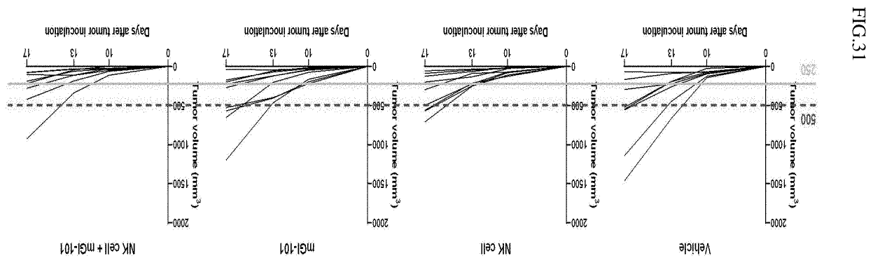

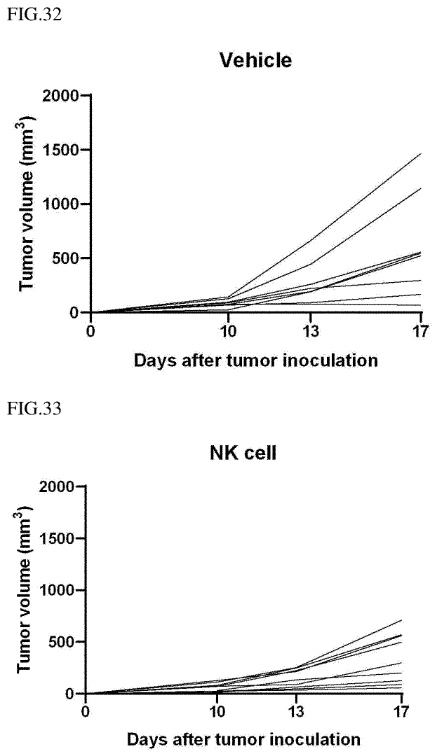

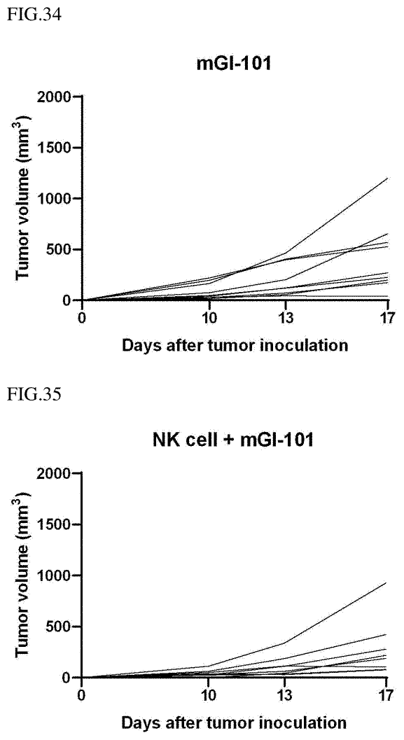

As described in Example 1 above, it was observed that respective test material GI-101 and CD80-Fc+Fc-IL2v2 did not significantly affect the viability of cancer cells when culturing cell lines for various cancer types (K562, MDA-MB-231, HCT-116, and A549 cell lines) in the presence of target cancer cells alone without NK cells (see Table 9, E/T ratio=0/1)(FIGS. 8 to 11).

### Example 1.2. Cancer Cell Proliferative Inhibition Effect of NK Cell and Fusion Protein Dimer Against K562 Cell (Lymphoblast)

The cancer cell killing capability of natural killer cells in a K562 cell line, a Leukemia cancer cell line, was confirmed. Specifically, the cancer cell viability according to treatment with a test material GI-101 or CD80-Fc+Fc-IL2v2 as a combination material when co-culturing K562 cell lines as target cells (T) and natural killer cells as effector cells (E) at E/T ratio=1/3, 1/1, 3/1 and 10/1, respectively was confirmed.

The result showed that when killing K562 cancer cells, treatment of GI-101 as a combination material of natural killer cell, which is a form of IL2-Fc-CD80 fusion protein, inhibited viability of cancer cells more than treatment of CD80-Fc+Fc-IL2v2 (FIGS. 16 to 19).

### Example 1.3. Cancer Cell Proliferative Inhibition Effect of NK Cell and Fusion Protein Dimer Against MDA-MB231 Cell

The cancer cell killing capability of natural killer cells in an MDA-MB231 cell line, a breast cancer cell line, was confirmed. Specifically, the cancer cell viability according to treatment with a test material GI-101 or CD80-Fc+Fc-IL2v2 as a combination material when co-culturing MDA-MB231 cell lines as target cells (T) and natural killer cells as effector cells (E) at E/T ratio=1/3, 1/1, 3/1 and 10/1, respectively was confirmed.

The result showed that when killing MDA-MB231 cancer cells, treatment of GI-101 as a combination material of natural killer cell, which is a form of IL2-Fc-CD80 fusion protein, inhibited viability of cancer cells more than treatment of CD80-Fc+Fc-IL2v2 (FIGS. 16 to 19).

### Example 1.4. Cancer Cell Proliferative Inhibition Effect of NK Cell and Fusion Protein Dimer Against HCT-116 Cell

The cancer cell killing capability of natural killer cells in an HCT-116 cell line, a colon cancer cell line, was confirmed. Specifically, the cancer cell viability according to treatment with a test material GI-101 or CD80-Fc+Fc-IL2v2 as a combination material when co-culturing HCT-116 cell lines as target cells (T) and natural killer cells as effector cells (E) at E/T ratio=1/3, 1/1, 3/1 and 10/1, respectively was confirmed.

The result showed that when killing HCT-116 cancer cells, treatment of GI-101 as a combination material of natural killer cell, which is a form of IL2-Fc-CD80 fusion protein, inhibited viability of cancer cells more than treatment of CD80-Fc+Fc-IL2v2 (FIGS. 20 to 23).

### Example 1.5. Cancer Cell Proliferative Inhibition Effect of NK Cell and Fusion Protein Dimer Against A549 Cell

The cancer cell killing capability of natural killer cells in a A549 cell line, a colon cancer cell line, was confirmed. Specifically, the cancer cell viability according to treatment with a test material GI-101 or CD80-Fc+Fc-IL2v2 as a combination material when co-culturing A549 cell lines as target cells (T) and natural killer cells as effector cells (E) at E/T ratio=1/3, 1/1, 3/1 and 10/1, respectively was confirmed.

The result showed that when killing A549 cancer cells, treatment of GI-101 as a combination material of natural killer cell, which is a form of IL2-Fc-CD80 fusion protein, inhibited viability of cancer cells more than treatment of CD80-Fc+Fc-IL2v2 (FIGS. 24 to 27).

### V. Determination of Cancer Cell Killing Capability of NK Cell and Fusion Protein Dimer in a Carcinoma Mouse Model: In Vivo

### Example 2. Administration of mGI-101 and NK Cell

To a carcinoma mouse model, the mGI-101 obtained in Preparation example 4 was administered by the intraperitoneal route and the mouse-derived NK cells prepared in Preparation example 3 were administered by the intravenous route. The administration was performed a total of three times once on the day of administration (day 6, day 10, and day 13 after tumor transplantation) using a disposable syringe (31G, 1 mL). For a tumor growth inhibition model, as shown in Table 10 below, cells to be administered and administration doses of the four groups were different (FIG. 28).

TABLE 10

| | | Dosage of hIgG4 | |
|---|---|---|---|
| | | or mGI- | Number of |
| Group | Cells administered | 101 (mg/kg) | NK cell |
| G1 (control) | hIgG4 | 4 | 0 |
| G2 | MgG4 + NK cell | 4 | 1 × 10⁶ |
| G3 | mGI101 alone | 0.6 | 0 |
| G4 | mGI101 + NK cell | 0.6 | 1 × 10⁶ |

### Example 3. Measurement of Tumor Volume of Carcinoma Mouse Model

Maximum length (L) and perpendicular width (W) of the tumor were measured twice a week using a Digital caliper (mitutoyo, Japan) and applied to Equation 1 below to calculate the tumor volume (TV).

TV( [symbol] ³)=W×W×L×0.5 [Equation 1]

The percentage of tumor growth inhibition was calculated by using the following Equation 2:

Tumor growth inhibition (%)=(1-(Ti-T0)/(Vi-V0))×100 [Equation 2]

Ti=tumor volume before administration in the test group

T0=tumor volume after administration in the test group

Vi=tumor volume before administration in the control group

V0=tumor volume after administration in the control group

The tumor volume of each individual before administration was set as the value measured at the time of grouping.

### Example 4. Determination of Tumor Growth Inhibitory Effect in the CT26 Transplanted Carcinoma Mouse Model

### Example 4.1. Measurement of Tumor Volume

The CT26 colorectal cancer cell suspension (5×10⁵ cells/0.1 mL) prepared for healthy Balb/c mice was dispensed, and the prepared solution was filled in a disposable syringe, and administered subcutaneously (0.1 mL/head) to the right back of the animal to transplant. After tumor transplantation, the drugs shown in Table 10 were administered, respectively. Then, the size of the tumor was measured on day, day 13, and day 17. Tumor growth was inhibited in the groups treated with natural killer cell (NK cell) or mGI-101 alone compared to the control group (vehicle). Tumor growth was inhibited in the group treated with natural killer cell (NK cell) in combination with mGI-101 compared to the control group (vehicle). Tumor growth of the group treated with natural killer cells in combination with mGI-101 was inhibited compared to the groups treated with natural killer cells or mGI-101 alone (FIG. 29).

### Example 4.2. Tumor Growth Inhibition Assay

Tumor growth inhibition rate was calculated at the end of the experiment (after tumor transplantation, day 17) compared to the drug treatment day 1 (after tumor transplantation, day 10). The control group (vehicle) had 3 mice having tumor growth inhibition rate of 30% or more, 3 mice having tumor growth inhibition rate of 50% or more, and 2 mice having tumor growth inhibition rate of 80%. The natural killer cell treatment group had 6 mice having tumor growth inhibition rate of 30% or more, 5 mice having tumor growth inhibition rate of 50% or more, and 2 mice having tumor growth inhibition rate of 80%. The mGI-101 treatment group had 5 mice having tumor growth inhibition rate of 30% or more, 5 mice having tumor growth inhibition rate of 50% or more, and 1 mouse having tumor growth inhibition rate of 80%. The natural killer cell and mGI-101 combination treatment group had 7 mice having tumor growth inhibition rate of 30% or more, 6 mice having tumor growth inhibition rate of 50% or more, and 3 mice having tumor growth inhibition rate of 80% (FIG. 30). In FIG. 30, a black bar indicates the tumor growth inhibition rate of 30% or more, a light gray bar indicates the tumor growth inhibition rate of 50% or more, and a dark gray bar indicates the tumor growth inhibition rate of 80% or more.

### Example 4.3. Measurement of Tumor Volume for Individual Laboratory Animals

Tumor growth of individual laboratory animals in each treatment group was determined. Specifically, tumor growth of individual laboratory animals in the control group (vehicle), natural killer cells (NK cells), mGI-101, and natural killer cells+mGI-101 treatment groups were determined and shown in FIG. 31. In FIG. 31, a dotted line indicates a tumor size of 500 mm³ and a solid line indicates a tumor size of 250 mm³.

More specifically, the degree of tumor growth of individual laboratory animals of the control group was determined and shown in FIG. 32, and the degree of tumor growth of individual laboratory animals of the natural killer cell treatment group was determined and shown in FIG. 33. In addition, the degree of tumor growth of individual laboratory animals of the mGI-101 treatment group was determined and shown in FIG. 34, and the degree of tumor growth of individual laboratory animals of the natural killer cells and mGI-101 combination treatment group was determined and shown in FIG. 35.

1. A method for treating cancer comprising:
administering a fusion protein to a subject in combination with a natural killer cell, wherein the fusion protein comprises an IL-2 protein and a CD80 protein.

2. The method for treating cancer according to claim 1, wherein the IL-2 protein and the CD80 protein are linked via a linker.

3. The method for treating cancer according to claim 1, wherein the IL-2 protein has the amino acid sequence of SEQ ID NO: 10.

4. The method for treating cancer according to claim 1, wherein the IL-2 protein is an IL-2 variant.

5. The method for treating cancer according to claim 4, wherein the IL-2 variant is obtained by substitution of at least one of the 38^{th}, 42^{nd}, 45^{th}, 61^{st}, and 72^{nd} amino acid in the amino acid sequence of SEQ ID NO: 10.

6. The method for treating cancer according to claim 4, wherein the IL-2 variant is obtained by at least one substitution selected from the group consisting of R38A, F42A, Y45A, E61R, and L72G, in the amino acid sequence of SEQ ID NO: 10.

7. The method for treating cancer according to claim 4, wherein the IL-2 variant comprises any one selected from the following substitution combinations (a) to (d) in the amino acid sequence of SEQ ID NO: 10:
(a) R38A/F42A
(b) R38A/F42A/Y45A
(c) R38A/F42A/E61R
(d) R38A/F42A/L72G.

8. The method for treating cancer according to claim 4, wherein the IL-2 variant has the amino acid sequence of SEQ ID NO: 6, 22, 23, or 24.

9. The method for treating cancer according to claim 1, wherein the CD80 protein has the amino acid sequence of SEQ ID NO: 11.

10. The method for treating cancer according to claim 1, wherein the CD80 protein is a CD80 fragment.

11. The method for treating cancer according to claim 10, wherein the CD80 fragment consists of the 35^{th} to 242^{nd} amino acids in SEQ ID NO: 11.

12. The method for treating cancer according to claim 2, wherein the linker is an albumin or an Fc domain of an immunoglobulin.

13. The method for treating cancer according to claim 12, wherein the Fc domain is a wild-type Fc domain or an Fc domain variant.

14. The method for treating cancer according to claim 12, wherein the Fc domain has the amino acid sequence of SEQ ID NO: 4.

15. The method for treating cancer according to claim 13, wherein the Fc domain variant has the amino acid sequence of SEQ ID NO: 12.

16. The method for treating cancer according to claim 1, wherein the fusion protein consists of the following structural formula (I) or (II):
N′—X—[linker(1)]ₙ-Fc domain-[linker(2)]ₘ-Y—C′ (I)
N′—Y-[linker(1)]ₙ-Fc domain-[linker(2)]ₘ-X—C′ (II)
wherein, in the structural formulas (I) and (II),
N′ is the N-terminus of the fusion protein,
C′ is the C-terminus of the fusion protein,
X is a CD80 protein,
Y is an IL-2 protein,
the linkers (1) and (2) are peptide linkers, and
n and m are each independently 0 or 1.

17. The method for treating cancer according to claim 16, wherein the linker (1) is a peptide linker consisting of the amino acid sequence of SEQ ID NO: 3.

18. The method for treating cancer according to claim 16, wherein the linker (2) is a peptide linker consisting of the amino acid sequence of SEQ ID NO: 5.

19. The method for treating cancer according to claim 16, wherein the fusion protein consists of the structural formula (I).

20. The method for treating cancer according to claim 1, wherein the fusion protein has a sequence identity of 85% or more to the amino acid sequence of SEQ ID NO: 9, 26, 28, or 30.

21. The method for treating cancer according to claim 1, wherein the fusion protein is a dimer.

22. The method for treating cancer according to claim 1, wherein the cancer is any one selected from the group consisting of gastric cancer, liver cancer, lung cancer, colorectal cancer, breast cancer, prostate cancer, ovarian cancer, pancreatic cancer, cervical cancer, thyroid cancer, larynx cancer, acute lymphoblastic leukemia, brain tumor, neuroblastoma, retinoblastoma, head and neck cancer, salivary cancer, and lymphoma.


SEQUENCE LISTING (47 sequences)

SEQ ID NO: 1 - protein, 25 aa, Artificial Sequence
  signal peptide (TPA)
MDAMLRGLCCVLLLCGAVFVSPSHA

SEQ ID NO: 2 - protein, 208 aa, Artificial Sequence [sequence omitted]

SEQ ID NO: 3 - protein, 30 aa, Artificial Sequence
  hinge with linker
GSGGGGSGGGGSGGGGSAESKYGPPCPPCP

SEQ ID NO: 4 - protein, 216 aa, Artificial Sequence
  immunoglobulin fc
APEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 5 - protein, 5 aa, Artificial Sequence
  linker
GGGGS

SEQ ID NO: 6 - protein, 133 aa, Artificial Sequence
  hIL-2M
APTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 617 aa, Artificial Sequence
  fusion protein comprising variants of IL-2 and fragments of CD80
MDAMLRGLCCVLLLCGAVFVSPSHAVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - DNA, 1857 bp, Artificial Sequence
  nucleotides coding fusion protein (GI101)
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacctgatga

SEQ ID NO: 9 - protein, 592 aa, Artificial Sequence
  fusion protein (GI101)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, Artificial Sequence [sequence omitted]

SEQ ID NO: 11 - protein, 288 aa, Artificial Sequence
  CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 12 - protein, 215 aa, Artificial Sequence
  modified Fc
SHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 306 aa, Artificial Sequence
  mCD80
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 14 - DNA, 1848 bp, Artificial Sequence
  nucleotides coding fusion protein (mGI101)
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtctcttggaggtggtggcggttctgcccctacctccagctctaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacttgcagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagagcatcatctccacactgacc

SEQ ID NO: 15 - protein, 616 aa, Artificial Sequence
  fusion protein (mGI101) with signal peptide
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - DNA, 1437 bp, Artificial Sequence
  nucleotides coding fusion protein (GI101C1)
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtccctgggc

SEQ ID NO: 17 - protein, 454 aa, Artificial Sequence
  fusion protein (GI101C1)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 18 - DNA, 1176 bp, Artificial Sequence
  nucleotides coding fusion protein (GI101C2)
atggatgctatgctgagaggcctgtgttgcgtgctgctgctgtgtggcgctgtgttcgtgtctccatctcacgccgctgagtctaagtacggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtctcttggcggaggcggaggatctgctcctacctccagctccaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacctccagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagtccatcatctccacactgacc

SEQ ID NO: 19 - protein, 367 aa, Artificial Sequence
  fusion protein (GI101C2)
AESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - DNA, 1434 bp, Artificial Sequence
  nucleotides coding fusion protein (mGI101C1)
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtccctgggc

SEQ ID NO: 21 - protein, 478 aa, Artificial Sequence
  fusion protein (mGI101C1)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 22 - protein, 133 aa, Artificial Sequence
  variants of IL-2 (3M, M45)
APTSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, Artificial Sequence
  variants of IL-2 (3M, M61)
APTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 24 - protein, 133 aa, Artificial Sequence
  variants of IL-2 (3M, M72)
APTSSSTKKTQLQLEHLLLDLQMILNGINNYKNPKLTAMLTAKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 25 - DNA, 1851 bp, Artificial Sequence
  nucleotides coding fusion protein (GI102-M45)
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttcgccatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 26 - protein, 592 aa, Artificial Sequence
  fusion protein (GI102-M45)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - DNA, 1851 bp, Artificial Sequence
  nucleotides coding fusion protein (GI102-M61)
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaaagggaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 28 - protein, 592 aa, Artificial Sequence
  fusion protein (GI102-M61)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 29 - DNA, 1857 bp, Artificial Sequence
  nucleotides coding fusion protein (GI102-M72)
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaagaagaactgaagcccctggaagaggtgctgaatggggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacctgatga

SEQ ID NO: 30 - protein, 592 aa, Artificial Sequence
  fusion protein (GI102-M72)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 31 - DNA, 1851 bp, Artificial Sequence
  nucleotides coding fusion protein (GI101w)
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 32 - protein, 592 aa, Artificial Sequence
  fusion protein (GI101w)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 33 - DNA, 1848 bp, Artificial Sequence
  nucleotides coding fusion protein (mGI102-M61)
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtctcttggaggtggtggcggttctgcccctacctccagctctaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacttgcagtgcctggaaagggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagagcatcatctccacactgacc

SEQ ID NO: 34 - protein, 616 aa, Artificial Sequence
  fusion protein (mGI102-M61)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 35 - protein, 153 aa, Artificial Sequence
  wild type hIL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - protein, 158 aa, Artificial Sequence
  IL-2 with signal sequence
MDAMLRGLCCVLLLCGAVFVSPSHAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 37 - DNA, 474 bp, Artificial Sequence
  nucleotide sequence coding IL-2 with signal sequence
atggatgctatgctgagaggcctgtgttgcgtgctgctgctgtgtggcgctgtgttcgtgtctccttctcacgctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 38 - protein, 13 aa, Artificial Sequence
  hinge
AESKYGPPCPPCP

SEQ ID NO: 39 - DNA, 1461 bp, Artificial Sequence
  nucleotides coding CD80-Fc protein
ggatccgccaccatggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtccctgggctgatgactcgag

SEQ ID NO: 40 - protein, 479 aa, Artificial Sequence
  CD80-Fc protein
MDAMLRGLCCVLLLCGAVFVSPSHAVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 41 - DNA, 1851 bp, Artificial Sequence; identical to SEQ ID NO: 31
  nucleotides coding fusion protein (hCD80-Fc- IL2wt)

SEQ ID NO: 42 - protein, 367 aa, Artificial Sequence
  Fc-IL2wt
AESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 43 - DNA, 1176 bp, Artificial Sequence
  Fc-IL2wt
atggatgctatgctgagaggcctgtgttgcgtgctgctgctgtgtggcgctgtgttcgtgtctccttctcacgctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 44 - protein, 392 aa, Artificial Sequence
  Fc-IL2v2
MDAMLRGLCCVLLLCGAVFVSPSHAAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 45 - DNA, 1200 bp, Artificial Sequence
  nucleotides coding Fc-IL2v2
ggatccgccaccatggatgctatgctgagaggcctgtgttgcgtgctgctgctgtgtggcgctgtgttcgtgtctccatctcacgccgctgagtctaagtacggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtctcttggcggaggcggaggatctgctcctacctccagctccaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacctccagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagtccatcatctccacactgacctgatgactcgag

SEQ ID NO: 46 - protein, 592 aa, Artificial Sequence; identical to SEQ ID NO: 32
  CD80-Fc-IL2wt

SEQ ID NO: 47 - protein, 591 aa, Artificial Sequence
  mGI-101
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT